(19) United States
(12) Patent Application Publication
(10) Pub. No.: US 2019/0298811 A1
(43) Pub. Date: Oct. 3, 2019

(54) COMPOUNDS, COMPOSITIONS AND METHODS FOR PREVENTING AND/OR TREATING INFLAMMATION AND/OR ORGAN DYSFUNCTION AFTER PEDIATRIC CARDIOVASCULAR SURGERY

(71) Applicant: THE REGENTS OF THE UNIVERSITY OF COLORADO, A BODY CORPORATE, DENVER, CO (US)

(72) Inventor: Jesse DAVIDSON, DENVER, CO (US)

(21) Appl. No.: 16/316,018

(22) PCT Filed: Jul. 5, 2017
(86) PCT No.: PCT/US17/40737

§ 371 Date: Jan. 7, 2019

Related U.S. Application Data
(60) Provisional application No. 62/359,075, filed on Jul. 6, 2016.
(60) Provisional application No. 62/514,628, filed on Jun. 2, 2017.

(51) Int. Cl. A61K 38/46; A61P 7/04

(52) U.S. Cl. CPC A61K 38/465; A61P 7/04; C12Y 301/03001

(57) ABSTRACT

The present invention includes a method of inhibiting and/or reducing blood coagulation in a subject. The present invention further provides a method of treating and/or preventing inflammation and/or organ dysfunction in a subject.

### CROSS-REFERENCE TO RELATED APPLICATIONS

The present application claims priority under 35 U.S.C. § 119(e) to U.S. Provisional Applications No. 62/359,075, filed Jul. 6, 2016, and No. 62/514,628, filed Jun. 2, 2017, all of which applications are incorporated herein by reference in their entireties.

### STATEMENT REGARDING FEDERALLY SPONSORED RESEARCH OR DEVELOPMENT

This invention was made with government support under UL1 RR025780 awarded by NIH/NCRR, 1K23HL123634 awarded by NIH/NHLBI, PR152240 awarded by DOD, and UL1 TR001082 awarded by NIH/NCATS. The government has certain rights in the invention.

### BACKGROUND OF THE INVENTION

Congenital cardiovascular defects are one of the most common birth defects (4-10 per 1000 live births). Surgical repair is often required during infancy and despite improving mortality, risk of death or transplant in the first year of life remains >25% for complex surgeries. Post-operative morbidity is also a significant concern: survivors often develop chronic heart failure, renal failure, hepatic dysfunction, intestinal injury, or neurologic injury.

Cardiopulmonary bypass (CPB), selective cerebral perfusion (SCP), and deep hypothermic circulatory arrest (DHCA) are often necessary for successful repair of congenital cardiovascular defects. However, these techniques independently result in global ischemia/reperfusion, inflammation, and organ injury/dysfunction. Understanding of these injury pathways is incomplete, and therapies to reduce post-surgical injury are largely limited to supportive care.

There is thus a need in the art for compositions and methods for treating and/or preventing inflammation and/or organ dysfunction in an infant after pediatric cardiovascular surgery. The present invention addresses these needs.

### BRIEF SUMMARY OF THE INVENTION

The invention provides a method of inhibiting and/or reducing blood coagulation in a human subject. The invention further provides a method of treating and/or preventing inflammation and/or organ dysfunction in a human subject. The invention further provides a method of reducing levels of extracellular adenine nucleotide in a human subject. The invention further provides a method of reducing levels of endotoxins in the blood of a human subject.

In certain embodiments, the method comprises administering to the subject a therapeutically effective amount of isolated human alkaline phosphatase (AP), and/or a biologically effective fragment, derivative, conjugate and/or recombinant form thereof. In other embodiments, the isolated human AP is the only therapeutic agent administered to the human subject. In yet other embodiments, the isolated human AP is the only therapeutic agent administered to the human subject in an amount sufficient to inhibit and/or reduce blood coagulation in the human subject, treat and/or prevent inflammation and/or organ dysfunction in the human subject, reduce levels of extracellular adenine nucleotide in the human subject, and/or reduce levels of endotoxins in the blood of the human subject.

In certain embodiments, the method comprises administering to the subject a therapeutically effective amount of isolated bovine alkaline phosphatase (AP), and/or a biologically effective fragment, derivative, conjugate and/or recombinant form thereof. In other embodiments, the isolated bovine AP is the only therapeutic agent administered to the human subject. In yet other embodiments, the isolated bovine AP is the only therapeutic agent administered to the human subject in an amount sufficient to inhibit and/or reduce blood coagulation in the human subject, treat and/or prevent inflammation and/or organ dysfunction in the human subject, reduce levels of extracellular adenine nucleotide in the human subject, and/or reduce levels of endotoxins in the blood of the human subject. In yet other embodiments, the isolated bovine AP is the only therapeutic agent administered to the human subject in an amount sufficient to treat and/or prevent organ dysfunction in the human subject.

In certain embodiments, activated clotting time (ACT) in the subject is increased. In other embodiments, clot rate (CT) in the subject is decreased.

In certain embodiments, the isolated human AP is at least one selected from the group consisting of intestinal, placental, placental-like, and tissue non-specific. In other embodiments, the isolated human AP is at least one selected from the group consisting of intestinal, placental, and tissue non-specific. In yet other embodiments, the isolated human AP is tissue non-specific. In yet other embodiments, the human tissue non-specific AP is at least one selected from the group consisting of liver, bone, and kidney. In yet other embodiments, the human tissue non-specific AP is human liver or bone AP. In yet other embodiments, the human tissue non-specific AP is human liver AP.

In certain embodiments, the isolated bovine AP is at least one selected from the group consisting of intestinal, placental, placental-like, and tissue non-specific. In other embodiments, the isolated bovine AP is at least one selected from the group consisting of intestinal, placental, and tissue non-specific. In yet other embodiments, the isolated bovine AP is tissue non-specific. In yet other embodiments, the bovine tissue non-specific AP is at least one selected from the group consisting of liver, bone, and kidney. In yet other embodiments, the bovine tissue non-specific AP is bovine liver or bone AP. In yet other embodiments, the bovine tissue non-specific AP is bovine liver AP. In yet other embodiments, the bovine AP is intestinal AP.

In certain embodiments, the subject has undergone and/or is undergoing cardiovascular surgery. In other embodiments, the subject has undergone and/or is undergoing cardiopulmonary bypass (CPB), such as but not limited to deep hypothermic circulatory arrest (DHCA). In yet other embodiments, the subject has undergone and/or is undergoing extracorporeal membrane oxygenation (ECMO). In yet other embodiments, the subject is an infant of about 120 days or less of age.

In certain embodiments, the isolated human AP is administered in a dose of about 4-150 U/kg. In other embodiments, the administration allows the subject to maintain a blood AP activity of at least about 80 U/L. In yet other embodiments, the administration allows the subject to maintain a blood AP activity of at least about 100-1,000 U/L.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates pre-operation to post-operation change in alkaline phosphatase activity versus change in 13C5-adenosine without CD73 inhibition. AP=alkaline phosphatase.

FIG. 2 illustrates pre-operation to post-operation change in alkaline phosphatase activity versus change in 13C5-adenosine with CD73 inhibition. AP=alkaline phosphatase.

FIG. 3 illustrates 13C5-adenosine production without ectonucleotidase inhibition, with CD73 inhibition, and with alkaline phosphatase inhibition. TNAP=tissue non-specific alkaline phosphatase.

FIG. 4 illustrates 13C5-adenosine production with ex vivo alkaline phosphatase supplementation. ⋄=mean. BiAP=bovine intestinal alkaline phosphatase; AP=alkaline phosphatase. Pairwise comparisons are presented in Table 4.

FIG. 5 comprises a series of graphs illustrating changes in isoform-specific alkaline phosphatase activity (pre-operative through 72 hours) for Example 2. 0=pre-operative; R=rewarming; 6=6 hours; 24=24 hours; 48=48 hours; 72=72 hours. Solid line=isoform-specific activity; dashed line=% total activity.

FIG. 6 illustrates a cell based model of coagulation monitored by Sonoclot (simplified overview).

FIGS. 7A-7B illustrate Sonoclot measurement of a cell based model of coagulation (ACT=initiation; CR=amplification and propagation; PF=clot retraction). Initiation: Liquid phase is characterized quantitatively with the Activated Clotting Time. Propagation: Fibrin formation continues at a near constant rate until available fibrinogen converts to fibrin. Amplification: Clot begins to rise at an increasing rate during amplification.

FIGS. 8A-8B are a series of graphs illustrating quantitative analysis of Sonoclot measurements. Sonoclot Signatures show multiple AP dose responses with increasing ACT and decreasing CR. AP had anticoagulable effects on every donor, although individual responses to AP dosage varied. Multiple donor responses shown herein. FIG. 8A, top: Control blood shown in gray (1), Red (2)=0.053 U/mL, Orange (3)=0.106 U/mL, Green (4)=0.160 U/mL, Light blue (5)=0.213 U/mL, Blue (6)=0.266 U/mL, Teal (7)=0.320 U/mL, Magenta (8)=0.400 U/mL, Purple (9)=0.480 U/mL. FIG. 8A, bottom: Control blood shown in gray (1), Green (4)=0.106 U/mL, Light blue (5)=0.160 U/mL, Blue (6)=0.213 U/mL, Magenta (8)=0.266 U/mL. FIG. 8B, top: Control blood shown in gray (1), Green (4)=0.106 U/mL, Blue (6)=0.160 U/mL, Teal (7)=0.213 U/mL, Purple (9)=0.266 U/mL. FIG. 8B, bottom: Control blood shown in gray (1), Green (4)=0.266 U/mL, Light blue (5)=0.400 U/mL, Dark Blue (6)=0.480 U/mL, Light Purple (9)=0.053 U/mL.

FIG. 9 comprises a series of graphs illustrating quantitative analysis for individual donors. Each of the donors displayed these same overall trends. The ACT and CR linear regression graphs show high R² values, indicating strong upward (ACT) and downward (CR) trends. The low R² value for PF indicates the lack of a strong upward or downward trend. The p values for both ACT and CR indicate the slope of the linear regression is significantly different from zero. The slope for PF was not significantly different from zero. For the Mean and Error graphs, Single-Factor ANOVAs were run to confirm/deny a significant difference between the activities of AP. ACT: There was a significant difference between the responses for 0 U/mL and 0.16 U/mL at the p<0.05 level [F(1,8)=64.5, p<0.0001]. CR: There was a significant difference between the responses for 0 U/mL and 0.16 U/mL at the p<0.05 level [F(1,8)=45.5, p=0.0001]. PF: There was no significant difference between the responses for 0 U/mL and 0.16 U/mL at the p<0.05 level [F(1,8)=0.076, p=0.79].

FIG. 10 comprises a graph illustrating mean CD73 pre and post-CPB.

FIG. 11 comprises a graph illustrating trend of mean CD73 level over time.

FIG. 12 comprises a bar graph illustrating median AP activity.

FIG. 13 comprises a bar graph illustrating mean endotoxin activity assay results.

FIG. 14 comprises a bar graph illustrating endotoxin activity assay results by pre-operative AP activity.

FIG. 15 comprises a bar graph illustrating endotoxin activity assay results by rewarming AP activity.

FIGS. 16A-16B comprise bar graph illustrating mean EAA with and without ex vivo liver AP supplementation [whole cohort] (FIG. 16A) pre-operative samples; (FIG. 16B) rewarming samples.

FIG. 17 illustrates an experimental design for an interventional study of bovine intestinal AP infusion in infant pigs undergoing cardiopulmonary bypass with DHCA as reported in Example 6.

FIG. 18 illustrate non-limiting gross pathology lung samples obtained according to Example 6. The samples are derived from subjects subjected to DHCA without therapeutic intervention (control samples).

FIG. 19 illustrate non-limiting gross pathology lung samples obtained according to Example 6. The samples are derived from subjects subjected to DHCA and treated with BiAP (75 U/kg bolus followed by continuous infusion of 25 U/kg/hr).

FIG. 20 illustrate non-limiting gross pathology lung samples obtained according to Example 6. Sample SID 10 is from a subject subjected to DHCA and treated with BiAP (75 U/kg bolus followed by continuous infusion of 25 U/kg/hr). Sample SID 9 is from a subject subjected to DHCA without therapeutic intervention (control sample). Sample SID 10 displayed much higher lung tissue air expansion, reduced interstitial thickening, and reduced inflammatory cell infiltrate than sample SID 9.

### DETAILED DESCRIPTION OF THE INVENTION

The present invention relates, in one aspect, to the unexpected discovery of various beneficial effects of the administration of isolated human alkaline phosphatase (AP), isolated bovine AP (such as but not limited to BiAP), and/or a biologically effective fragment, derivative, conjugate and/or recombinant form thereof, to infants undergoing cardiovascular surgery. A decrease in AP is observed in infants after pediatric cardiac surgery, and this decrease is associated with increase morbidity after surgery, as well as increased procalcitonin (a biomarker of systemic inflammation). Further, the decrease in AP activity is secondary to a loss of protein (rather than altered activity of existing protein), and this corresponds to an across-the-board loss of AP (rather than a specific isoform). This loss is associated with subsequent requirements for ICU support, and increased organ dysfunction (renal and cardiac) and injury (intestinal). AP can clear extracellular AMP (the most important soluble ectonucleotidase), and addition of AP to serum ex vivo improves clearance of AMP. Further, data included herein shows a clear decrease in endotoxin produced during pediatric cardiac surgery with the addition of AP ex vivo.

Alkaline phosphatases (APs) are a family of endogenous metalloenzymes, with multiple isoforms, present in serum and most organs. AP has demonstrated beneficial physiologic activities in vitro. These activities include dephosphorylation of endotoxin to a less toxic monophosphoryl byproduct and conversion of harmful extracellular adenine nucleotides to adenosine. Preclinical and Phase II adult studies of AP therapy for sepsis and inflammatory colitis have shown a reduction in inflammation and organ injury.

As demonstrated herein, studies were performed to evaluate the association between AP activity and serum conversion of adenosine monophosphate (AMP) to adenosine after infant cardiac surgery, as well as assess if inhibition/supplementation of serum AP modulates this conversion. With that objective, pre/post-bypass serum samples were obtained from 75 infants <4 months of age. Serum conversion of 13C5-adenosine monophosphate to 13C5-adenosine was assessed with/without selective inhibition of AP and CD73. Low and high dose 13C5-adenosine monophosphate (simulating normal/stress concentrations) were used. Effects of AP supplementation on adenosine monophosphate clearance were also assessed. Changes in serum AP activity were strongly correlated with changes in 13C5-adenosine production with or without CD73 inhibition (r=0.83; p<0.0001). Serum with low AP activity (≤80 U/L) generated significantly less 13C5-adenosine, particularly in the presence of high dose 13C5-adenosine monophosphate (10.4 μmol/L vs 12.9 μmol/L; p=0.0004). Inhibition of AP led to a marked decrease in 13C5-adenosine production (11.9 μmol/L vs 2.7 μmol/L; p<0.0001). Supplementation with physiologic dose human tissue non-AP or high dose bovine intestinal AP doubled 13C5-adenosine monophosphate conversion to 13C5-adenosine (p<0.0001).

AP represents the primary serum ectonucleotidase after infant cardiac surgery and low post-operative AP activity leads to impaired capacity to clear adenosine monophosphate. AP supplementation improves serum clearance of adenosine monophosphate to adenosine. These findings represent a therapeutic mechanism for AP infusion during cardiac surgery. AP was thus identified as the primary soluble ectonucleotidase in infants undergoing cardiopulmonary bypass, and decreased capacity to clear AMP when AP activity decreases post-bypass was demonstrated. Supplementation of AP ex vivo improves this capacity and can represent the beneficial therapeutic mechanism of AP infusion seen in phase 2 studies.

As demonstrated herein, studies were performed to determine the kinetics of AP activity and concentration after infant cardiopulmonary bypass including isoform-specific changes, as well as to measure the association between low post-operative AP activity and major post-operative cardiovascular events, organ injury/dysfunction, and post-operative support requirements. With that objective, a prospective cohort study of 120 infants ≤120 days of age undergoing cardiopulmonary bypass was studied. AP total and isoform-specific activity was assessed at 6 time-points (pre-operation, rewarming, 6, 24, 48, and 72 h post-operation). Low AP activity was defined as ≤80 U/L. AP concentrations as well as biomarkers of organ injury/dysfunction were collected through 24 h post-operation. Major cardiovascular events were defined as cardiac arrest, mechanical circulatory support, or death.

AP activity loss occurred primarily during the operation (median decrease 89 U/L; p<0.0001) secondary to decreased bone and liver 2 isoforms. Activity declined through 24 h in 27% of patients. AP activity strongly correlated with serum concentration (r=0.87-0.91; p<0.0001). Persistent low AP activity at 72 h was independently associated with occurrence of a major cardiac event (OR 5.6; p<0.05). Early AP activity was independently associated with subsequent vasoactive-inotropic score (p<0.001), peak lactate (p<0.0001), peak creatinine (p<0.0005), NT-proBNP (p<0.05), and intestinal fatty acid binding protein (p<0.005).

It was found that_AP activity decreases during infant cardiopulmonary bypass and may continue to drop for 24 h. Activity loss is secondary to decreased bone and liver 2 isoform concentrations. Early low AP activity is independently associated with subsequent post-operative support and organ injury/dysfunction, while persistence of AP activity ≤80 U/L at 72 h is independently associated with increased odds of major cardiovascular events.

### Definitions

As used herein, each of the following terms have the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics and chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, ±5%, ±1%, or ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound of the invention, or a derivative, solvate, salt or prodrug salt thereof, along with a compound and/or composition that may also treat the disorders or diseases contemplated within the invention. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound and/or composition which is sufficient to provide a beneficial effect to the subject to which the compound and/or composition is administered. The term to “treat,” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of the composition and/or compound of the invention in a kit.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

“Parenteral” administration of a composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound and/or composition useful within the invention with other chemical components, such as carriers, stabilizers, diluents, dispersing agents, suspending agents, thickening agents, and/or excipients. The pharmaceutical composition facilitates administration of the compound and/or composition to an organism. Multiple techniques of administering a compound and/or composition exist in the art including, but not limited to: intravenous, oral, aerosol, parenteral, ophthalmic, pulmonary, intracranial and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt, prodrug, solvate or derivative of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, a “subject” refers to a human or non-human mammal. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a composition useful within the invention (alone or in combination with another pharmaceutical agent), to a subject, or application or administration of a therapeutic agent to an isolated tissue or cell line from a subject (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder or the potential to develop the disease or disorder. Such treatments may be specifically tailored or modified, based on knowledge obtained from the field of pharmacogenomics.

The following abbreviations are used herein: ADA1, adenosine deaminase 1; ADP, adenosine diphosphate; AMP, adenosine monophosphate; AP, alkaline phosphatase; ATP, adenosine triphosphate; BiAP, bovine intestinal alkaline phosphatase; CICU, cardiac intensive care unit; CPB, cardiopulmonary bypass; DHCA, deep hypothermic circulatory arrest; EHNA, erythro-9-(2-hydroxy-3-nonyl)adenine; ENT, 1,2 Equilibrative nucleoside transporter 1 and 2; iFABP, intestinal fatty acid binding protein; LOS, length of stay; NGAL, neutrophil gelatinase associated lipocalin; NT-proBNP, n-terminal pro-brain natriuretic peptide; OR, odds ratio; SCP, selective cerebral perfusion; TNAP, tissue non-specific alkaline phosphatase.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods

The invention provides a method of inhibiting and/or reducing blood coagulation in a subject. The invention further provides a method of treating and/or preventing inflammation and/or organ dysfunction in a subject. The invention further provides a method of reducing levels of extracellular adenine nucleotide in a subject. The invention further provides a method of reducing levels of endotoxins in the blood of a subject.

In certain embodiments, the method comprises administering to the subject a therapeutically effective amount of isolated human alkaline phosphatase (AP), and/or a biologically effective fragment, derivative, conjugate and/or recombinant form thereof.

In certain embodiments, the method comprises administering to the subject a therapeutically effective amount of isolated bovine alkaline phosphatase (AP), and/or a biologically effective fragment, derivative, conjugate and/or recombinant form thereof. In other embodiments, the method of treating and/or preventing organ dysfunction in a subject comprises administering to the subject a therapeutically effective amount of isolated bovine alkaline phosphatase (AP), and/or a biologically effective fragment, derivative, conjugate and/or recombinant form thereof. In yet other embodiments, the bovine AP is BiAP.

In certain embodiments, the subject has undergone and/or is undergoing cardiovascular surgery. In other embodiments, the administration takes place before, during or after the cardiovascular surgery.

In certain embodiments, the subject has undergone and/or is undergoing cardiopulmonary bypass (CPB). In other embodiments, the subject has undergone and/or is undergoing extracorporeal membrane oxygenation (ECMO).

In certain embodiments, the subject is an infant of about 120 days or less of age.

In certain embodiments, the activated clotting time (ACT) in the subject is increased. In other embodiments, the clot rate (CT) in the subject is decreased.

In certain embodiments, the human AP is at least one selected from the group consisting of intestinal, placental, placental-like, and tissue non-specific. In certain embodiments, the human AP is at least one selected from the group consisting of intestinal, placental, and tissue non-specific. In other embodiments, the human AP is tissue non-specific.

In certain embodiments, the human tissue non-specific AP is at least one selected from the group consisting of liver AP, bone AP, and kidney AP. In other embodiments, the human tissue non-specific AP is at least one selected from the group consisting of liver AP and bone AP. In yet other embodiments, the human tissue non-specific AP is liver AP.

In certain embodiments, the human AP is administered a dose varying from about 4 U/kg to about 150 U/kg.

In certain embodiments, the administration allows the subject to maintain a blood AP activity of at least about 80 U/L. In other embodiments, the administration allows the subject to maintain a blood AP activity of at least about 100-1,000 U/L.

In certain embodiments, the compound and/or composition of the invention is administered by an inhalational, oral, rectal, vaginal, parenteral, intracranial, topical, transdermal, pulmonary, intranasal, buccal, ophthalmic, intrathecal, or intravenous route of administration.

### Compositions and Combination Therapies

The invention contemplates the use of any isolated human alkaline phosphatase (AP), and/or a biologically effective fragment, derivative, conjugate and/or recombinant form thereof. In a non-limiting example, the invention contemplates the use of recombinant human AP (recAP-hybrid human intestinal/placental AP) designed for therapeutic use.

In certain embodiments, the compounds and/or compositions contemplated within the invention are useful within the methods of the invention in combination with at least one additional agent useful for treating or preventing a disease or disorder contemplated herein. This additional compound may comprise compounds identified herein or compounds, e.g., commercially available compounds, known to treat or prevent a disease or disorder contemplated herein.

A synergistic effect may be calculated, for example, using suitable methods such as, for example, the Sigmoid-Eₘₐₓ equation (Holford & Scheiner, 1981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.1 and 5,000 mg/kg (or 0.1 and 5,000 U/kg) per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician could start doses of the compounds of the invention employed in the pharmaceutical composition at levels lower than that required in order to achieve the desired therapeutic effect and gradually increase the dosage until the desired effect is achieved.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier.

In certain embodiments, the compositions of the invention are administered to the patient by continuous infusion and/or in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in humans. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Unless otherwise noted, all cell lines, starting materials and reagents were obtained from commercial suppliers and used without purification.

### Example 1: Alkaline Phosphatase, Soluble Extracellular Adenine Nucleotides, and Adenosine Production after Infant Cardiopulmonary Bypass

The potential therapeutic mechanisms of AP in cardiac surgery settings are unclear. All APs are capable of hydrolytic phosphatase activity targeting a variety of molecules. Perhaps the most intriguing targets are extracellular adenine nucleotides released through cellular apoptosis or necrosis during ischemia/reperfusion injury. Extracellular adenosine triphosphate (ATP), adenosine diphosphate (ADP), and adenosine monophosphate (AMP) lead to inflammatory activation, vasoconstriction, and platelet activation, while stepwise dephosphorylation of these substrates to adenosine may be protective. In healthy neonates, AP serves as the primary serum enzyme responsible for the conversion of extracellular AMP to adenosine.

Given this function of AP in the healthy neonate, it is possible that the decrease in AP activity routinely seen after infant cardiothoracic surgery can lead to a reduced serum capacity to clear extracellular adenine nucleotides. It was also investigated whether AP continues to be the primary serum ectonucleotidase in this pathologic setting and that whether capacity to clear AMP to adenosine can be directly linked to residual AP activity. Together, these results can help identify a key mechanism linking low AP activity to inflammation and increased cardiovascular support requirements after infant cardiothoracic surgery. In addition, while AP therapy has demonstrated promising early results in multiple diseases, the effects of therapeutic AP administration on adenine nucleotide clearance and adenosine production are unknown. Therefore, the ex vivo effects of AP supplementation on AMP clearance were investigated to evaluate if AP has the potential to modulate this pathway as part of its therapeutic mechanism.

Study Design and Participants

This study was performed as a primary aim of a prospective cohort study assessing the biology and kinetics of AP in infants undergoing surgical repair or palliation of congenital heart disease. Inclusion criteria were age ≤120 days at the time of surgery and the use of CPB for repair. Patients were excluded if they were less than 34 weeks corrected gestational age or weighed less than 2 kg at the time of surgery in order to avoid excessive research blood draws and risk of anemia.

Sample Collection and Processing

Serum samples were obtained from indwelling arterial or venous catheters after induction of anesthesia and prior to first surgical incision (“pre-operative”). A portion of the sample was used for assessment of serum AP activity while the remaining serum was stored frozen at −70° C. for batch analysis of AMP to adenosine conversion. A second serum sample was obtained from each subject during the rewarming phase of CPB with identical processing (“post-operative”).

Alkaline Phosphatase Activity Assays

AP activity was determined on each sample using a clinically available photometric p-nitrophenol phosphate cleavage assay (Mayo Medical Laboratories, Rochester Minn.). Briefly, AP cleaves p-nitrophenol phosphate in the presence of magnesium to yield phosphate and n-nitrophenol. The rate of p-nitrophenol production (determined photometrically at 450 nm) is directly proportional to AP activity. Low AP activity in this population was defined a priori as ≤80 U/L.

AMP/Adenosine Conversion Assays

Stored serum was assessed via HPLC-MS/MS for conversion of exogenous 13C5-AMP to 13C5-adenosine (both Toronto Research Chemicals, Toronto, ON, Canada). Low (5 μmol/L; patients 1-25) and high (50 μmol/L; patients 26-50) dose 13C5-AMP were used to simulate normal/stress concentrations of AMP. Reactions were terminated after 15 minutes by addition of five volumes acetonitrile/methanol containing 5 μmol/L dl-adenosine as internal standard. In all assays, adenosine deaminase 1 (ADA1) and equilibrative nucleoside transporter 1 and 2 (ENT1,2) were blocked with erythro-9-(2-hydroxy-3-nonyl)adenine (EHNA) and dipyridamole respectively [Tocris Cookson; Bristol, UK] in order to prevent adenosine breakdown/reuptake respectively, thus trapping the adenosine allowing accurate measurement of its production. Native AMP and adenosine levels were also measured to assess background signal.

Alkaline Phosphatase Inhibition/Supplementation

13C-AMP conversion to adenosine was assessed with and without selective inhibition of tissue non-specific AP (TNAP) and CD73, the other major soluble ectonucleotidase. TNAP inhibition was accomplished using MLS-0038949 [EMD Millipore (Billerica, Mass., USA)], and CD73 inhibition through addition of adenosine 5′-(α,β-methylene)-diphosphate [Sigma (St. Louis, Mo., USA)].

The final set of assays sought to determine if addition of exogenous AP can increase 13C5-AMP clearance to 13C5-adenosine in the presence of high concentrations of 13C5-AMP (50 μmol/L). AP was tested in two forms: bovine intestinal AP (BiAP) (Alloksys Life Sciences B.V., Bunnik, NL) and human liver AP (MyBioSource, San Diego, Calif.). BiAP is the primary form used for therapeutic trials in both intestinal and systemic disease, making testing of this potential therapeutic mechanism clinically relevant. Human liver AP was also tested in this setting to evaluate if additive benefit can be derived by supplementing with one of the isoforms most abundant in serum under typical conditions. Five dosing strategies were chosen: no supplementation, low dose BiAP (500 U/L), low dose BiAP+low dose liver AP (both at concentrations of 500 U/L), high dose BiAP (50,000 U/L), and high dose BiAP+low dose liver AP. ADA1 and ENT1/2 were inhibited in all samples. CD73 was not inhibited for these assays as we sought to demonstrate increased adenosine production above the total baseline serum ectonucleotidase capacity.

Statistical Analysis

Patients' demographics and baseline clinic characteristics were summarized using mean and standard deviation for continuous variables, while frequency and percent were used for dichotomized variables. Two-sample t-test, Chi-square/Fisher's exact test as appropriate were performed to compare the difference between groups. Spearman's correlation test was performed to evaluate the correlation between AP total activity and adenosine product at pre-operation and pre-separation time points. Paired t-test was performed to compare the ratio of 13C5-adenosine production with and without CD73 inhibition. ANOVA method and paired t-tests were employed to compare adenosine production by various AP supplementation strategies. All the data analyses were performed using SAS V9.4, and graphs were polished in GraphPad Prism 6.0 using the output data from SAS.

### Selected Results:

Subjects

The first 76 subjects enrolled in the cohort were included in this arm of the study. One was excluded from the analysis due to insufficient serum sample volume. Baseline characteristics are presented in Table 1. The cohort was typical for the overall infant population in our system, demonstrating a high percentage for neonates with critical heart disease, high comprehensive Aristotle scores, and one quarter of patients exhibiting single ventricle physiology. A quarter of patients required mechanical ventilation at some stage during the pre-operative period and 12% required pre-operative inotropic support. Surgical variables included a wide range of CPB and aortic cross clamp times with relatively frequent use of either deep hypothermic circulatory arrest or selective cerebral perfusion.

AP Activity

A total of 72 subjects had AP activity analyzed in both the pre-operative and rewarming samples. AP activity could not be determined in one pre-operative sample and two rewarming samples due to interference from gross hemolysis. Mean total AP activity in the cohort decreased from 199.5 U/L pre-operation to 103.9 U/L during rewarming (p<0.0001). Pre-operative AP activity was low in three subjects (4%) based on the a priori definition of AP activity ≤80 U/L. At the rewarming stage, this number increased to 24 subjects (33%).

AP Activity Versus Adenosine Production

To assess the importance of total AP activity for determining serum capacity to clear AMP to adenosine, the correlation of AP activity to 13C5-adenosine production was examined under different experimental conditions. These results are shown in Table 2. Overall, total AP activity showed a strong correlation with 13C5-adenosine production at the pre-operative time point. The correlation was higher in the high dose 13C5-AMP group compared to the low dose 13C5-AMP group, suggesting that AP activity can be a more important determinant of AMP clearance at higher concentrations of AMP. CD73 inhibition increased the correlation of AP activity with 13C5-adenosine production at low concentrations of 13C5-AMP, but did not change the correlation when the higher concentration 13C5-AMP was used.

Post-operation the correlation between AP activity and 13C5-adenosine production decreased (Table 2). This decrease was most profound in the low concentration 13C5-AMP group without CD73 inhibition, but the decrease was present under all conditions.

It was investigated if post-operative serum samples with low AP activity (≤80 U/L) demonstrate decreased capacity to clear 13C5-AMP to 13C5-adenosine. Post-operative serum from infants with low AP activity generated significantly less 13C5-adenosine when challenged with high concentration 13C5-AMP (12.9 μmol/L vs 10.4 μmol/L; p<0.0005). A similar trend was present that did not reach statistical significance for post-operative serum challenged with low concentration 13C5-AMP (2.0 μmol/L vs 1.9 μmol/L; p=0.13). Comparing samples in the upper and lower quartiles of AP activity, a larger difference in adenosine production was observed when challenged with high concentration 13C5-AMP (14.2 μmol/L vs 9.7 μmol/L; p<0.005) and a similar trend with low concentration 13C5-AMP (2.1 μmol/L vs 1.8 μmol/L; p=0.08). When CD73 was inhibited, serum from infants with low AP activity produced significantly less 13C5-adenosine when challenged with either high or low dose 13C5-AMP (8.7 μmol/L vs 7.7 μmol/L; p<0.05 and 1.4 μmol/L vs 1.1 μmol/L; p<0.005 respectively). These differences were more marked when the lowest and highest quartiles were compared (10.1 μmol/L vs 7.5 μmol/L; p<0.005 and 1.6 μmol/L vs 1.1 μmol/L; p<0.01 respectively).

Change in AP Activity Versus Change in Adenosine Production

It was then evaluated if decreased AP activity across time points (pre-operative to post-operative) correlated directly with the change in serum capacity to convert AMP to adenosine. In serum challenged with low concentration 13C5-AMP, a statistically significant correlation between change in AP activity and change in 13C5-adenosine production (r=0.50; p<0.05) was observed. This correlation increased modestly with addition of CD73 inhibition (r=0.57; p<0.005). In serum challenged with high concentration 13C5-AMP, the correlation between change in AP activity and change in 13C5-adenosine production was much stronger with (r=0.84; p<0.0001) or without (r=0.83; p<0.0001) CD73 inhibition.

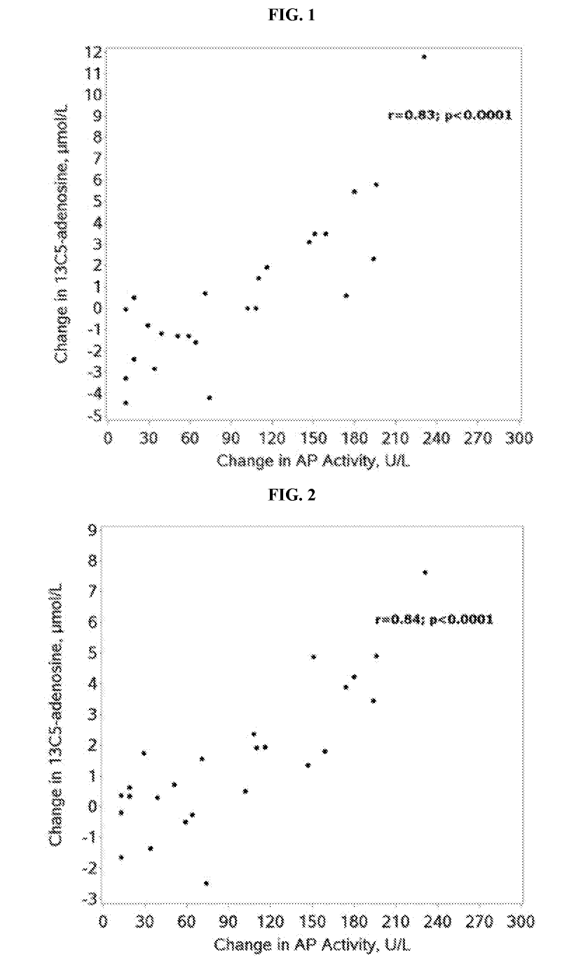

While changes in AP activity appeared to be the primary driver of changes in 13C5-adenosine, 13C5-adenosine production did not decrease in all subjects post-operatively despite decreases in AP activity in all subjects (FIG. 1). In subjects with minimal change in AP activity between the pre-operative and post-operative time points, a rise in 13C5-adenosine production was observed at the post-operative time point, which was substantially blunted by inhibition of CD73 (FIG. 2). CD73 inhibition did not, however, appear to change the overall association of AP activity and 13C5-adenosine production, suggesting a relatively constant effect of CD73 across all post-operative samples. While limits on the volume of blood drawn for research did not allow for directly assessing this unexpected finding of potentially increased CD73 activity post-operatively, the importance of CD73 was indirectly assessed through comparison of the ratio of 13C5-adenosine production with and without CD73 inhibition in pre and post-operative samples. These results are shown in Table 3. Across the entire cohort, this ratio decreased substantially post-operatively, indicating a more prominent role for CD73 post-operatively. CD73 appeared to play a more substantial role at lower concentrations of 13C5-AMP with a less prominent effect at high 13C5-AMP concentrations

AP Inhibition

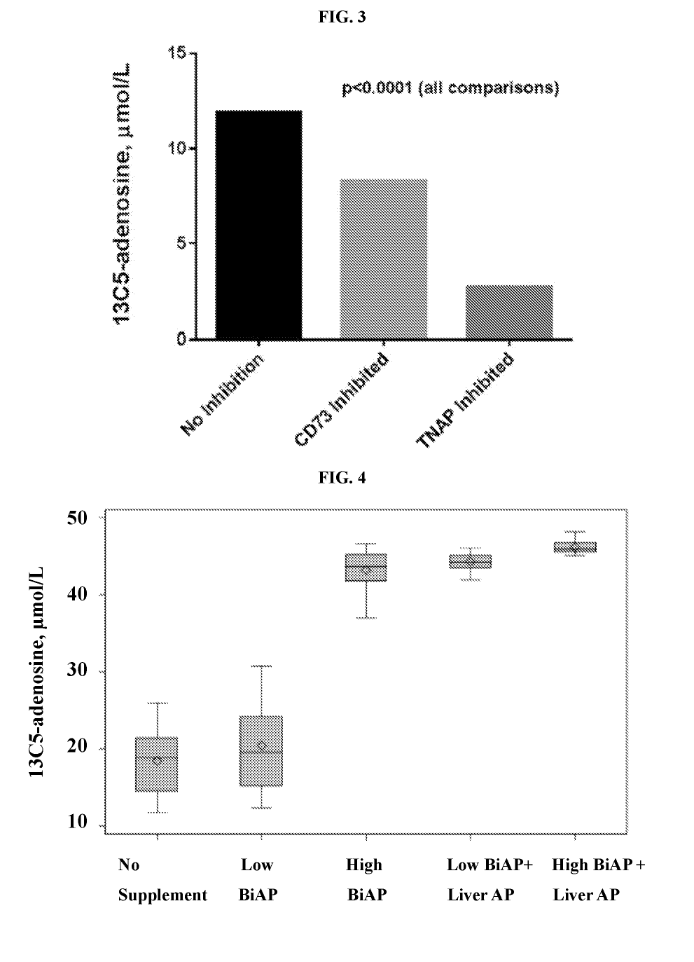

The strong correlation of serum AP activity to serum 13C5-adenosine production is suggestive that AP is the primary serum ectonucleotidase in the present patient population. To directly test the contribution of AP to adenosine production, a known selective inhibitor of TNAP (MLS-0038949) was used to block AP activity in the post-operative samples challenged with high concentration 13C5-AMP (FIG. 3). Inhibition of TNAP led to a marked decrease in 13C5-adenosine production (11.9 μmol/L vs 2.7 μmol/L; p<0.0001). In comparison, selective inhibition of CD73 was significantly less effective than TNAP inhibition for blocking 13C5-adenosine production (8.3 μmol/L vs 2.7 μmol/L; p<0.0001).

AP Supplementation

On average only 40% of 13C5-AMP added at low concentration and 25% of 13C5-AMP added at high concentration was converted to 13C5-adenosine in serum from the first 50 subjects, leaving a substantial concentration of 13C5-AMP in the serum at the time of reaction termination. One of the proposed beneficial mechanisms of AP therapy in disease is increased clearance of AMP and other extracellular adenine nucleotides to adenosine. Therefore, effects of the addition of exogenous AP on serum capacity to clear 13C5-AMP to adenosine were explored. Initial testing was performed using BiAP, as this is the formulation currently under evaluation in therapeutic trials for sepsis, ulcerative colitis, and cardiac surgery. Subsequently human liver AP, one of the isoforms typically present in human serum, wsd added to assess for additive effects.

Results of these assays are shown in FIG. 4 and Table 4. Across all groups, AP supplementation resulted in a highly statistically significant increase in 13C5-adenosine production (p<0.0001). Comparison among different AP regimens was also performed, testing for significance at the 0.05 level adjusted for multiple comparisons. Addition of low dose BiAP (500 U/L) resulted in a modest increase in 13C5-adenosine of 1.9 μM relative to baseline that did not reach statistical significance. Increasing BiAP to high dose (50,000 U/L) resulted in a statistically significant increase in 13C5-adenosine production (mean increase 24.4 μmol/L). With this level of BiAP activity, ˜90% of the 13C5-AMP was converted to 13C5-adenosine. Unexpectedly, use of human liver AP at physiologic levels (500 U/L) in addition to BiAP had a dramatic additive effect on 13C5-adenosine production. Addition of physiologic liver AP to low dose BiAP increased adenosine production relative to baseline or low dose BiAP alone (25.5 and 23.6 μmol/L respectively). Furthermore, 13C5-adenosine production with this regimen was comparable to that obtained by the use of supraphysiologic BiAP (44.4 vs 43.7 μmol/L). Addition of physiologic liver AP to high dose BiAP resulted in a smaller but statistically significant increase in 13C5-adenosine production when compared to high dose BiAP alone (2.9 μmol/L).

Relatively little is known about the physiologic role of AP despite conservation of these enzymes from bacteria to man. Missense mutations in the tissue nonspecific form of AP in humans result in failure to hydrolyze PPi and the severe clinical syndrome of hypophosphotasia. Other functions of serum and tissue-based AP are less well understood.

The present study is the first to evaluate the role of soluble AP on the clearance of AMP in a disease state. The findings of a strong positive correlation between AP activity and adenosine production as well as a five-fold reduction in adenosine production with selective AP blockade indicate that AP is the primary soluble ectonucleotidase in the setting of infant cardiopulmonary bypass. It was also confirmed a significantly lower adenosine production in serum with AP activity ≤80 U/L, providing support to clinical findings of worse clinical outcomes in infants with low post-operative AP activity.

Two additional findings regarding the role of native soluble AP in AMP clearance warrant further discussion. First, the relative importance of AP ectonucleotidase activity appears to increase with higher concentrations of AMP. At physiologic concentrations of AMP, CD73 acts as the dominant soluble ectonucleotidase, with AP taking a more prominent role as AMP increases to levels simulating concentrations found in pathologic states. The correlation between AP activity and adenosine production was higher at higher concentrations of AMP, and the relative contribution of CD73 was lower when more AMP was available as a substrate.

Next, in post-operative serum soluble AP demonstrates a reduced role in the conversion of AMP to adenosine. Part of this diminished role for AP can be directly explained by the marked decrease in AP activity after surgery. Serum AP activity decreases after cardiac surgery with cardiopulmonary bypass in both children and adults. A profound decrease in AP activity was observed after infant cardiopulmonary bypass (mean serum AP activity fell 48% post-operation). Indirect evidence from the study, however, suggests that part of the reduced role for AP in adenosine production may come from an increase in CD73 activity. Limitations on blood volume drawn for research prevented direct measurement of CD73 in this set of patients. However, patients with small decreases in AP activity post-operatively actually demonstrated an increase in adenosine production. This increase was largely eliminated with CD73 inhibition, suggesting that an increase in CD73 might be responsible for these findings.

TABLE 1
Clinical Characteristics

| | | | Post-op AP | | Post-op AP | | |
|---|---|---|---|---|---|---|---|
| | cohort | | Activity ≤80 U/L | | Activity >80 U/L | | p-value ≤80 |
| | N = 75 | | N = 24 | | N = 49 | | vs >80 |
| Male (%) | 43 | (57.3%) | 14 | (58.3%) | 27 | (55.1%) | 0.79 |
| Age at surgery, days; | 18 | (1, 119) | 4.5 | (1, 108) | 46 | (4, 119) | <0.0001 |
| median[range] | | | | | | | |
| Weight at surgery, kg; | 3.5 | (2.2, 7.2) | 3.1 | (2.2, 4.9) | 4.0 | (2.3, 7.2) | <0.001 |
| median[range] | | | | | | | |
| Pre-operation | 20 | (27.4%) | 8 | (33.3%) | 11 | (23.4%) | 0.37 |
| intubation (%) | | | | | | | |
| Pre-operation | 9 | (12.0%) | 3 | (12.5%) | 6 | (12.2%) | 1.0 |
| inotropic/vasoactive | | | | | | | |
| support (%) | | | | | | | |
| Pre-operation | 40 | (55.6%) | 19 | (79.2%) | 20 | (43.5%) | <0.005 |
| steroids (%) | | | | | | | |
| Single ventricle | 18 | (24.0%) | 8 | (33.3%) | 9 | (18.4%) | 0.15 |
| physiology (%) | | | | | | | |
| Aristotle Score- | 8.5 | (3.0, 15.0) | 10 | (3, 15) | 8 | (3, 14.5) | <0.05 |
| Comprehensive; | | | | | | | |
| median[range] | | | | | | | |
| Cardiopulmonary | 122 | (54, 399) | 148.5 | (75, 399) | 119 | (54, 215) | <0.01 |
| bypass time, minutes; | | | | | | | |
| median[range] | | | | | | | |
| Cross-clamp time, | 69 | (0, 241) | 80 | (0, 241) | 62 | (0, 142) | <0.005 |
| minutes; | | | | | | | |
| median[range] | | | | | | | |
| Deep hypothermic | 0 | (0, 77) | 7.5 | (0, 77) | 0 | (0, 59) | 0.001 |
| circulatory arrest, | | | | | | | |
| minutes; | | | | | | | |
| median[range] | | | | | | | |
| Selective cerebral | 0 | (0, 115) | 0 | (0, 115) | 0 | (0, 37) | 0.12 |
| perfusion, minutes; | | | | | | | |
| media[range] | | | | | | | |

TABLE 2
Correlation of AP activity to 13C5-adenosine production pre-
operation and prior to separation from cardiopulmonary bypass

| | |
|---|---|
| Pre-operative | Post-operative |

r (p-value)

| Low dose 13C-AMP | | | |
|---|---|---|---|
| Without CD73 Inhibition | 0.70 (<0.0001) | 0.48 | (<0.05) |
| With CD73 Inhibition | 0.82 (<0.0001) | 0.80 | (<0.0001) |
| High dose 13C-AMP | | | |
| Without CD73 Inhibition | 0.87 (<0.0001) | 0.76 | (0.0001) |
| With CD73 Inhibition | 0.88 (<0.0001) | 0.70 | (0.0001) |

TABLE 3
Ratio of 13C5-adenosine production with and without
CD73 inhibition, pre-operation and prior to separation
from cardiopulmonary bypass

| | Pre- | Post- | Pre-Post | |
|---|---|---|---|---|
| | operative, | operative, | Mean Difference | |
| | mean [SD] | mean [SD] | (95% CI) | p-value |
| Entire Cohort | 0.75 (0.09) | 0.68 (0.08) | 0.08 (0.05, 0.11) | <0.0001 |
| Low Dose | 0.72 (0.10) | 0.65 (0.09) | 0.07 (0.03, 0.10) | <0.005 |
| 13C5-AMP | | | | |
| High Dose | 0.79 (0.07) | 0.70 (0.07) | 0.09 (0.04, 0.13) | <0.001 |
| 13C5-AMP | | | | |

TABLE 4
Adenosine production with AP supplementation;

| | Difference Between | 95% Confidence | Significance |
|---|---|---|---|
| Group Comparison | Means (μmol/L) | Limits | at 0.05 level |
| No supplement vs. | 1.9 | (−0.8, 4.7) | |
| low dose BiAP | | | |
| No supplement vs. | 24.4 | (21.6, 27.2) | + |
| high dose BiAP | | | |
| No supplement vs. | 25.5 | (22.7, 28.3) | + |
| low dose BiAP + low dose liver AP | | | |
| No supplement vs. | 27.4 | (24.6, 30.1) | + |
| high dose BiAP + low dose liver AP | | | |
| Low dose BiAP vs. | 22.5 | (19.7, 25.3) | + |
| high dose BiAP | | | |
| Low dose BiAP vs. | 23.6 | (20.8, 26.3) | + |
| low dose BiAP + low dose liver AP | | | |
| Low dose BiAP vs. | 25.4 | (22.6, 28.2) | + |
| high dose BiAP + low dose liver AP | | | |
| High dose BiAP vs. | 1.1 | (−1.7, 3.9) | |
| low dose BiAP + low dose liver AP | | | |
| High dose BiAP vs. | 2.9 | (0.1, 5.7) | + |
| high dose BiAP + low dose liver AP | | | |
| Low dose BiAP + low dose liver AP vs. | 1.8 | (−0.9, 4.6) | |
| high dose BiAP + low dose liver AP | | | |

+ = significant at the 0.05 level adjusted for multiple comparisons

Assessment of native AP activity relative to clearance of AMP is an important finding that helps explain a portion of the variation in post-operative course for infants undergoing cardiothoracic surgery. The present study is the first to evaluate the effect of exogenous AP administration on extracellular adenine nucleotide clearance capacity in human serum. AP administration in serum with high concentrations of added 13C5-AMP was used to demonstrate relevance at concentrations representing stressed states. BiAP and human liver AP were tested to evaluate addition of both the therapeutic form and the native form of AP. Both BiAP and human liver AP were able to provide added clearance of 13C5-AMP above that accomplished by the native ectonucleotidases in this model, supporting this effect as a mechanism of action of therapeutic AP.

One unexpected finding was the difference in the magnitude of effect with human liver AP compared to BiAP. While the study was not primarily designed to assess different dosing regimens for AP, roughly similar potency from BiAP and human liver AP were anticipated. Instead a roughly 100-fold increased potency for human liver AP compared to BiAP was identified. These findings suggest that human AP can be effective at physiologic doses, whereas BiAP may require supraphysiologic dosing to have similar effects. Without wishing to be limited by any theory, the reasons behind this difference may include differences in half-life, species or isoform specific action on AMP, and differential clearance of bovine versus human proteins. The recently produced recombinant human AP (recAP-hybrid human intestinal/placental AP) designed for therapeutic use is also useful within the present methods.

### Example 2: Alkaline Phosphatase in Infant Cardiopulmonary Bypass: Kinetics and Relationship to Organ Injury and Major Cardiovascular Events

In certain embodiments, AP can play an important role after cardiothoracic surgery. AP activity decreases after cardiothoracic surgery in adults and children. In addition, low post-operative serum AP activity is independently associated with increased post-operative support requirements in infants. However, significant gaps exist in the understanding of AP after infant cardiothoracic surgery, including the timing and persistence of decreased AP activity, isoform-specific changes, the balance of enzyme loss versus deactivation, and the association with major cardiovascular events as well as organ injury.

The present study was designed to address these knowledge gaps. In certain non-limiting embodiments, loss of AP activity can begin during surgery and continue through the initial post-operative period. Activity of all AP isoforms could be equally affected and reflect decreased serum concentration rather than enzyme deactivation. In other non-limiting embodiments, early low AP activity could be associated with subsequent increased odds of a major cardiovascular event, increased post-operative support requirements, and increased evidence of renal, intestinal, and cardiac injury/dysfunction.

The study described herein was a prospective, observational cohort study of infants ≤120 days of age undergoing cardiothoracic surgery with CPB. Exclusion criteria were weight <2 kg (limited blood volume) and adjusted gestational age <34 weeks (altered AP production).

The primary study aims were to define the post-operative kinetics of AP, assess the correlation of AP activity to AP concentration, and determine if low post-operative AP activity is associated with an increased risk of cardiac arrest, mechanical circulatory support, or death. Low post-operative AP was defined a priori as ≤80 U/L. Secondary aims included: 1) assessment of isoform-specific AP changes, 2) validation of the association between AP activity and post-operative support requirements, and 3) measurement of the association between AP activity and organ injury/dysfunction.

CPB was performed using a neonatal circuit consisting of a roller head pump (S5, LivaNova, Arvada, Colo., USA) and a Terumo FX05 oxygenator with a blood prime. The blood prime routinely underwent pre-bypass hemofiltration using a Minntech Hemocor HPH Junior hemoconcentrator (Medivators Inc., Minneapolis, Minn., USA) with a polysulfone membrane prior to initiating bypass, allowing for partial filtration of molecules up to 65,000 Daltons. Anticoagulation was achieved prior to CPB by administering 500 units/Kg of heparin systemically to the patient. Initial target flow rate was approximately 200 ml/Kg/minute. Cardioplegia was accomplished using del Nido formula cardioplegia solution at an initial dose of 30 ml/Kg, and subsequent dosing was considered after 60 minutes of aortic cross-clamp time.

Baseline clinical information was obtained from all subjects (Table 5). Serum samples were obtained pre-operatively, with rewarming from CPB, and at 6, 24, 48, and 72 h after arrival in the cardiac intensive care unit (CICU). The 48 h sample was not performed in subjects ≤3 kg to limit blood draw volumes. Also, to minimize discomfort the protocol did not allow venipuncture. Therefore, no additional samples were obtained after removal of indwelling catheters.

Laboratory Analyses

Samples for AP activity were frozen to −70° C.; total and isoform specific AP activity were analyzed using commercially available assays (Mayo Laboratories, Rochester, Minn.). Total AP activity was measured using a standard photometric p-nitrophenol phosphate cleavage assay. AP cleaves p-nitrophenol phosphate to yield phosphate and n-nitrophenol. The rate of p-nitrophenol production (determined photometrically at 450 nm) is directly proportional to AP activity. Isoforms were separated using electrophoresis with additional isoform sialyation to achieve separation between liver and bone isoforms. Isoform activity was then determined using the specific chromogenic substrate, 5-bromo-4-chloro-3-indolyl phosphate/nitro blue tetrazolium in combination with densitometric quantification.

AP concentration and organ-specific injury/functional biomarkers were measured through 24 h using multiplex immunoassays (Meso Scale Diagnostics, Gaithersburg, Md.). Biomarkers included N-terminal pro b-type natriuretic peptide (NT-proBNP-cardiac function), intestinal fatty acid binding protein (iFABP-enterocyte injury), and neutrophil gelatinase-associated lipocalin (NGAL-proximal renal tubule injury). Peak creatinine and lactate were also recorded from post-operative measurements obtained as standard of care.

Clinical Outcomes

The primary clinical outcome was occurrence of any of the following major cardiovascular events: 1) cardiac arrest, 2) unplanned post-operative mechanical circulatory support, or 3) death (within 30 days or during the primary hospitalization). Secondary clinical outcomes included vasoactive inotropic score (VIS) at 6, 24, 48, and 72 h post-operation, time to first extubation, and CICU/hospital length of stay (LOS).

Statistics

Subjects' baseline characteristics were summarized using descriptive statistics. Correlation between AP activity and concentration was assessed by Spearman correlation test. Spearman correlation test was performed to select candidate predictors for multivariable modeling. Distributions of continuous outcomes were inspected prior to modeling; natural log transformation was applied as indicated. General linear model was used for log transformed outcomes and logistic regression was performed for dichotomized outcomes. Initial potential covariates were selected based on clinical or biological significance. Covariates that were significantly associated with both the primary explanatory variable and the outcome on univariate analysis were included in the initial model. Backwards stepwise regression was used to arrive at the final model. P value <0.05 was considered statistically significant. SAS V9.4 (SAS Institute, Cary, N.C.) was used for data management and analyses; GraphPad Prism 6 was used for plots.

### Results:

One hundred and twenty-two subjects enrolled in the study. Two subjects were converted to non-bypass surgery after enrollment and excluded. Subjects 1-100 were enrolled consecutively from the first 120 eligible patients (83% enrollment rate, Table 6). The final 22 subjects were enrolled from eligible morning surgeries only to allow processing time for pilot laboratory tests. Baseline clinical and surgical data are presented in Table 5.

AP Activity and Kinetics

AP activity was obtained in 98.8% of possible time points through 24 h post-operation (Table 7). Median AP activity through 72 h post-operation is shown in Table 8. AP activity decreased substantially between the pre-operative and rewarming samples (median decrease 89 U/L; p<0.0001). AP activity continued to fall in 61% of subjects between rewarming and 6 h, and 27% demonstrated further decline between 6 and 24 h. Low AP activity (≤80 U/L) was found in 5% of infants pre-operation, 30% at rewarming, 40% at 6 h, and 34% at 24 h. A comparison of baseline characteristics for infants with AP activity ≤80 U/L vs. >80 U/L at the rewarming time point is presented in Table 5. After 24 h, AP activity rose consistently with only rare exceptions. The mean increase between 24 and 72 h was 26.9 U/L (p<0.0001). This change likely underestimates the rise for the whole cohort as we did not obtain 72 h levels in a subset of healthier infants who no longer had indwelling catheters (Table 9). Sixteen infants demonstrated AP activity ≤80 U/L at 72 h. Only a minority of infants (9%) regained pre-operative activity levels by 72 h.

Isoform-Specific Activity

Isoform-specific activity is shown in FIG. 5. Bone and liver 2 isoforms represented the clear majority of AP prior to surgery. Bone and liver 2 AP activities fell between pre-operation and rewarming, were stable to slightly decreased by 6 h, and started slowly rising by 24 h. Intestinal AP activity doubled between pre-operative and rewarming samples but remained a small portion of total AP activity. The liver 1 isoform increased markedly and constituted 17% of total AP activity at rewarming. Liver 1 activity was maintained through 6 h then subsequently declined.

Concentration Versus Activity

Serum AP activity and concentration were compared to assess if changes in AP activity could be attributed primarily to decreased AP concentration. AP concentrations declined from pre-operation to rewarming, and then gradually increased after 24 h. Activity and concentration were strongly correlated (r=0.87-0.91; p<0.0001), suggesting protein loss rather than enzyme dysfunction as the primary cause of diminished AP activity.

Clinical Outcomes

The primary clinical outcome was the odds of a major cardiovascular event in subjects with AP activity ≤80 U/L. Eleven subjects experienced a major cardiovascular event (9%). On univariate analysis, subjects with low AP activity at 6, 24, or 72 h demonstrated increased odds of experiencing a major event (6 h Odds Ratio [OR] 4.4, p<0.05; 24 h OR 3.8, p<0.05; 72 h OR 8.7, p<0.005). On multivariable analysis, low AP activity at 72 h remained statistically significant (OR 5.6; p<0.05), with the other time points demonstrating an OR of 2 but with confidence intervals crossing 1. The absolute risk of a major event in subjects with a 72 h AP activity ≤80 U/L was 30%. The major cardiovascular events appeared to separate into two groups. Eight subjects experienced sudden cardiac arrest or need for mechanical circulatory support and all demonstrated an AP activity of ≤80 U/L at the time point prior to the event. The remaining three subjects maintained AP activity >80 U/L but had progressive decline or withdrawal of support.

Secondary clinical outcomes included post-operative VIS, length of intubation, CICU/hospital LOS (Table 10). AP activity demonstrated an independent negative association with VIS at all time points on multivariable modeling. AP was most strongly associated with concurrent VIS. In the models, each 10 U/L decrease in AP at the 6 hr time point was associated with a 6% increase in VIS 6 hr (p<0.0001), while for each 10 U/L decrease in AP at the 24 hr time point, VIS at 24 hr increased by 7% (p<0.0001). Of note, AP activity at the rewarming time point (prior to the initiation of inotropic and vasoactive support) was also independently associated with subsequent VIS at 6 and 24 hrs. AP activity at 24 h was independently associated with length of intubation (4% increase in length of intubation for each 10 U/L decrease in AP; p<0.05), with a similar trend for AP activity at 6 h that did not reach statistical significance. AP activity was not independently associated with CICU or hospital LOS.

It was assessed if early AP activity (rewarming and 6 h) was associated with biomarkers of organ perfusion, injury, and dysfunction. AP activity at 6 h was associated with higher subsequent (24 h) NT-proBNP and iFABP levels but not concurrent (6 h) levels of these markers (Table 7). AP activity at 6 h also demonstrated a strong independent association with peak creatinine and peak lactate (Table 11). Looking earlier, AP activity at rewarming also showed a strong, independent negative association with peak creatinine and peak lactate, but not NT-proBNP or iFABP (Table 11). There was no association between AP activity and NGAL. However, in the cohort, NGAL also showed no association with peak creatinine or change in creatinine, so it is unclear how well the biomarker performed in our specific population.

Selected Comments

This study represents the first comprehensive examination of AP activity after infant CPB. The key findings center on a significant early loss of AP activity that began prior to separation from bypass, and was attributable to decreased concentrations of bone and liver 2 isoforms. Most subjects showed ongoing AP loss through 6 h post-operation, and nearly a third experienced continued decline through 24 h. Early low AP activity demonstrated a strong, independent association with subsequent cardiovascular support requirements, peak lactate, and peak creatinine. Early low AP activity was also associated with subsequent biomarkers of enterocyte injury and cardiac dysfunction. Recovery of AP activity typically began within 72 h, however only a small minority of subjects regained their pre-operative AP activity by that time. Failure to increase AP activity above 80 U/L by 72 h was an ominous sign, with this subset of subjects demonstrating increased odds of a major cardiovascular event.

In the present cohort, most AP activity loss also occurred early after surgery. Ongoing decreases were observed through 6 and even 24 h post-operation, indicating the process is not isolated to the operation. Different from adult cohorts, AP activity in the presently examined infants remained well below pre-operative levels through 72 h, demonstrating a more profound deficiency and potentially slower resolution of the underlying mechanism.

The exact etiology of AP activity loss after cardiac surgery has yet to be defined. The present study provides the first evidence that decreased AP activity is secondary to decreased serum concentration. There appears to exist no correlation between AP activity and total protein levels. Filtration is also unlikely as AP is larger than the filtration size of the circuit, and AP activity decreases in some cases of off-bypass infant cardiac surgery. Decreased production of AP has been proposed, however patients receiving a systemic infusion of bovine intestinal AP during CPB demonstrate preservation of native AP activity. Without wishing to be limited by any theory, this finding is more consistent with consumption than decreased production. A consumptive process is also supported by animal models suggesting AP targets endotoxin released during intestinal ischemia-reperfusion, forming a conjugate that is subsequently cleared by Kupffer cells in the liver.

Isoform-specific changes lend additional insight. The present findings demonstrate a proportional decrease in bone and liver 2 isoforms during infant cardiac surgery. This proportional decrease suggests either decreased production of both isoforms or more likely a consumptive process. In contrast, liver 1 and intestinal isoforms were not detectable pre-operation but rose post-operatively.

From a clinical standpoint, AP fell prior to separation from bypass, temporally preceding key short term clinical outcomes. Early low AP activity was strongly associated with subsequent cardiovascular instability and poor oxygen delivery as evidenced by higher VIS and peak lactate levels. The worst cases of cardiovascular instability (post-operative cardiac arrest or unplanned mechanical circulatory support requirement) were uniformly preceded by low AP activity, and persistently low AP activity at 72 h was associated with increased odds of experiencing a major cardiovascular event. Low AP activity also associated with and temporally preceded biomarker evidence of renal, enterocyte, and cardiac injury/dysfunction. Low AP activity was not associated with CICU or hospital LOS, indicating that the changes observed were limited to the early postoperative period.

The timing of decreased AP activity is important. AP can be protective after ischemia-reperfusion injury and other inflammatory processes through the dephosphorylation of toxic phosphate-containing molecules. Two proposed targets of AP, endotoxin and extracellular adenine nucleotides, are released following ischemia-reperfusion injury and can lead to cardiovascular instability, tissue injury, and inflammation. There is a decrease in serum capacity to convert extracellular adenosine monophosphate (AMP) to adenosine in the immediate post-operative period that is directly related to serum AP activity. Ex-vivo addition of liver AP markedly improves this capacity. Without wishing to be limited by any theory, decreased AP activity can result in poor clearance of endotoxin and/or extracellular adenine nucleotides, leading to subsequent inflammation and cardiovascular instability. The fact that AP activity declines early in the perioperative course makes it amenable to therapeutic intervention. A phase 2 study of bovine intestinal AP infusion during coronary artery bypass grafting demonstrated decreased inflammation, although no difference in outcomes in this low risk patient population, indicating that this treatment is feasible and may have clinical efficacy.

Aa demonstrated herein, AP activity decreases markedly during infant cardiac surgery with CPB and can continue to drop through 24 h post-operation. Activity loss is secondary to decreased bone and liver 2 isoform concentrations. Early low AP activity is independently associated with subsequent increased post-operative support and organ injury/dysfunction, while persistence of AP ≤80 U/L at 72 h is independently associated with increased odds of cardiac arrest, mechanical circulatory support, or death.

TABLE 5
Demographics, baseline clinical characteristics, and comparison between infants with alkaline
phosphatase activity ≤80 U/L vs. >80 U/L at rewarming from cardiopulmonary bypass

| Baseline and Surgical | | | Rewarming AP | | Rewarming AP | | p-value ≤80 |
|---|---|---|---|---|---|---|---|
| Characteristics | Full Cohort | | activity ≤80 U/L | | Activity >80 U/L | | vs. >80 |
| Male (%) | 68 | (55.7%) | 21 | (58.3%) | 44 | (53.7%) | NS |
| Age at surgery, days; | 15 | (1, 120) | 5.5 | (1, 120) | 45 | (2, 119) | <0.0001 |
| median[range] | | | | | | | |
| Preterm (%) | 16 | (13.2%) | 4 | (11.1%) | 12 | (14.8%) | NS |
| Weight; median[range] | 3.5 | (2.1, 7.9) | 3.2 | (2.2, 4.9) | 3.8 | (2.1, 7.9) | <0.005 |
| Pre-operative mechanical | 39 | (32.2%) | 14 | (38.9%) | 24 | (29.3%) | NS |
| ventilation (%) | | | | | | | |
| Pre-operative inotropic support (%) | 15 | (12.3%) | 3 | (8.3%) | 12 | (14.6%) | NS |
| Aristotle Score*- | 10 | (3, 19.5) | 11.8 | (3. 16) | 9 | (3, 19.5) | <0.005 |
| Comprehensive; median[range] | | | | | | | |
| Aristotle Score*-Basic; | 9 | (3, 15) | 10.5 | (3, 15) | 9 | (3, 14.5) | <0.005 |
| Median[range] | | | | | | | |
| Cardiopulmonary bypass time, | 137 | (0, 399) | 161.5 | (75, 399) | 126.5 | (54, 323) | <0.005 |
| minutes; median[range] | | | | | | | |
| Cross-clamp time, minutes; | 74 | (0, 241) | 82 | (0, 241) | 68.5 | (0, 188) | <0.05 |
| median[range] | | | | | | | |
| Deep hypothermic circulatory | 0 | (0, 154) | 6 | (0, 77) | 0 | (0, 154) | <0.0001 |
| arrest, minutes; median[range] | | | | | | | |
| Selective cerebral perfusion, | 0 | (0, 115) | 0 | (0, 82) | 0 | (0, 115) | <0.005 |
| minutes; media[range] | | | | | | | |
| Single ventricle physiology (%) | 38 | (31.7%) | 17 | (48.6%) | 20 | (24.4%) | <0.05 |
| Perioperative steroid | 65 | (55.6%) | 29 | (80.6%) | 36 | (44.4%) | <0.005 |
| administration (%) | | | | | | | |

*Jacobs, et al., 2008, Cardiol Young 18 Suppl 2: 163-8

TABLE 6
Non-enrolled patients

| Number | Reason for not enrolling |
|---|---|
| 1 | Urgent/Emergent |
| 2 | Competing study |
| 3 | Declined |
| 4 | Competing study |
| 5 | Competing study |
| 6 | Urgent/Emergent |
| 7 | Urgent Transplant |
| 8 | No available study personnel |
| 9 | Urgent Transplant |
| 10 | No available study personnel |
| 11 | Declined |
| 12 | Parents not available for consent |
| 13 | Urgent/Emergent |
| 14 | Declined |
| 15 | Declined |
| 16 | Declined |
| 17 | Parents not available for consent |
| 18 | Ward of the State |
| 19 | Emergent Surgery |
| 20 | Emergent Surgery |

TABLE 7
Missed alkaline phosphatase samples

| Study ID Number | Time Point | Reason for Missed Sample |
|---|---|---|
| 6 | 6 hour | Quantity not sufficient |
| 9 | 6 hour | Quantity not sufficient |
| 15 | 24 hour | Indwelling catheters removed |
| 20 | Rewarming | Gross hemolysis |
| 57 | Pre-operative | Gross hemolysis |
| 73 | Rewarming | Separation from bypass prior to sample |
| | | collection |

TABLE 8
Alkaline phosphatase activity

| | | Median AP | | |
|---|---|---|---|---|
| | | Activity | | |
| Time Point | n | (U/L) | IQR | Range |
| Pre-operation | 120 | 184 | 128, 273 | 55, 618 |
| Rewarming | 118 | 97 | 75, 133 | 34, 315 |
| 6 Hour | 118 | 92 | 65, 121 | 32, 302 |
| 24 Hour | 119 | 99 | 70, 147 | 32, 310 |
| 72 Hour | 95 | 124 | 97, 171 | 42, 293 |

TABLE 9
Comparison of baseline and surgical characteristics of subjects with
and without 72 h blood draw for alkaline phosphatase activity.

| | | | 72 Hour AP activity | | | |
|---|---|---|---|---|---|---|
| Baseline and Surgical Characteristics | Full cohort | | No | | Yes | |
| Male (%) | 16 | (55.7%) | 16 | (59.3%) | 52 | (54.7%) |
| Age at surgery, days; median[range] | 15 | (1, 120) | 68 | (2, 119) | 9 | (1, 120) |
| Preterm % | 16 | (13.2%) | 4 | (14.8%) | 12 | (12.8%) |
| Weight | 3.5 | (2.1, 7.9) | 4.3 | (2.1, 7.2) | 3.5 | (2.1, 7.9) |
| Pre-operative intubation | 39 | (32.2%) | 4 | (15.4%) | 35 | (36.8%) |
| Pre-operative inotropic support | 15 | (12.3%) | 1 | (3.7%) | 14 | (14.7%) |
| Aristotle Score-Comprehensive; | 10 | (3, 19.5) | 8 | (3, 14.5) | 10.2 | (3, 19.5) |
| median[range] | | | | | | |
| Cardiopulmonary bypass time, minutes; | 137 | (0, 399) | 99.5 | (0, 274) | 147 | (54, 399) |
| median[range] | | | | | | |
| Cross-clamp time, minutes; median[range] | 74 | (0, 241) | 60 | (0, 147) | 75 | (0, 241) |
| Deep hypothermic circulatory arrest, | 0 | (0, 154) | 0 | (0, 38) | 1 | (0, 154) |
| minutes; median[range] | | | | | | |
| Selective cerebral perfusion, minutes; | 0 | (0, 115) | 0 | (0, 115) | 0 | (0, 82) |
| media[range] | | | | | | |
| Single ventricle physiology (%) | 38 | (31.7%) | 3 | (11.5%) | 35 | (37.2%) |
| Steroid | 65 | (55.6%) | 9 | (34.6%) | 58 | (61.7%) |
| VIS 6 hour; median[range] | 9 | (0, 27) | 5 | (0, 25) | 9 | (2.5, 27.0) |
| VIS 24 hour; median[range] | 7 | (0, 30) | 3 | (0, 30) | 8 | (0, 26) |
| Intubation, hours; median[range] | 44.4 | (0.1, 762.6) | 23 | (0.1, 453.1) | 50.9 | (3.2, 762.5) |
| ICU length of stay, days; median[range] | 6 | (1, 42) | 2 | (1, 28) | 7 | (1, 42) |

TABLE 10
Multivariable analysis of post-operative alkaline
phosphatase activity and post-operative support

| | | Increase in | |
|---|---|---|---|
| | | Dependent Variable | |
| AP Time | Dependent Variable | per 10 U/L Decrease | |
| Point | (Outcome) | in AP Activity | p-value |
| Rewarming | VIS 6 h | 3% | <0.05 |
| | VIS 24 h | 4% | <0.05 |
| | Length of Intubation | 2% | NS |
| | ICU Length of Stay | 2% | NS |
| | Hospital Length of Stay | 3% | NS |
| 6 hour | VIS 6 h | 6% | <0.0001 |
| | VIS 24 h | 4% | <0.05 |
| | Length of Intubation | 4% | NS |
| | ICU Length of Stay | 1% | NS |
| | Hospital Length of Stay | 3% | NS |
| 24 hour | VIS 24 h | 7% | <0.0001 |
| | Length of Intubation | 4% | <0.05 |
| | ICU Length of Stay | 1% | NS |
| | Hospital Length of Stay | 2% | NS |

TABLE 11
Multivariable analysis of post-operative alkaline
phosphatase activity and organ injury/dysfunction

| | | Increase in | |
|---|---|---|---|
| | | Dependent Variable | |
| AP Time | | per 10 U/L Decrease | |
| Point | Dependent Variable | in AP Activity | p-value |
| Rewarming | iFABP 6 h | 1% | NS |
| | NT-proBNP 6 h | 0% | NS |
| | Peak Creatinine | 2% | <0.0005 |
| | Peak Lactate | 7% | <0.0005 |
| | NGAL 6 h | 2% | NS |
| 6 hour | iFABP 6 h | 10% | NS |
| | iFABP 24 h | 12% | <0.005 |
| | NT-proBNP 6 h | −1% | NS |
| | NT-proBNP 24 h | 6% | <0.05 |
| | Peak Creatinine | 4% | <0.005 |
| | Peak Lactate | 9% | <0.0001 |
| | NGAL 6 h | 0% | NS |

### Example 3: AP Alters Clot Initiation, Amplification and Propagation in Healthy Human Adult Donors

AP dephosphorylates various types of molecules, including endotoxin, nucleotide phosphates, and pro-coagulant polyphosphates (polyp). AP activity falls after CV surgery and low levels are associated with increased post-operative support and organ injury/dysfunction. Persistent low AP levels at 72 hrs post-op is independently associated with increased odds of cardiac arrest, mechanical circulatory support, or death. AP's mechanism of protection and its effects on coagulation are currently unknown. Viscoelastic instrumentation such as the Sonoclot (SCP2) can show changes in hemostasis by utilizing real time sample clot formation monitoring.

The present study has in part the objective of determining the effect of AP on hemostasis in healthy human adult donors using the SCP2 to provide coagulation factor specific results, focusing on activated clotting time (ACT) (clot initiation) and clot rate (CR) (clot amplification and propagation).

Citrated whole blood was collected from adult healthy volunteers and spiked with various concentrations of AP. Two different sources of human AP, recombinant bone (0.008 μg/mL-0.053 μg/mL) and liver (0.053 U/mL-0.40 U/mL activity), were utilized. Blood was re-calcified and run on the SCP2 using the mildly activated gbACT+ test. Results for ACT, CR, and platelet function were analyzed.

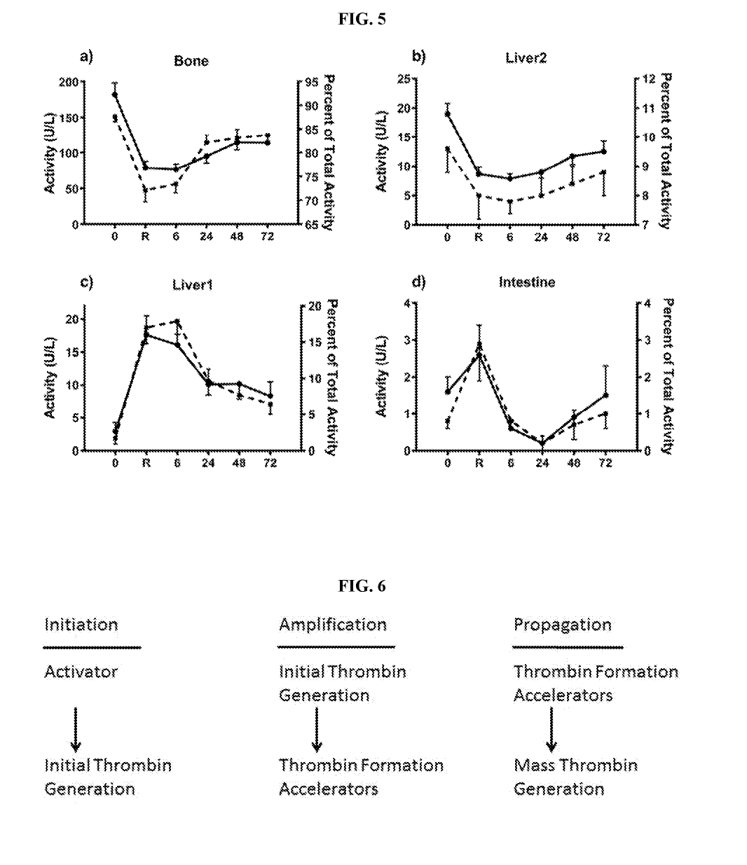

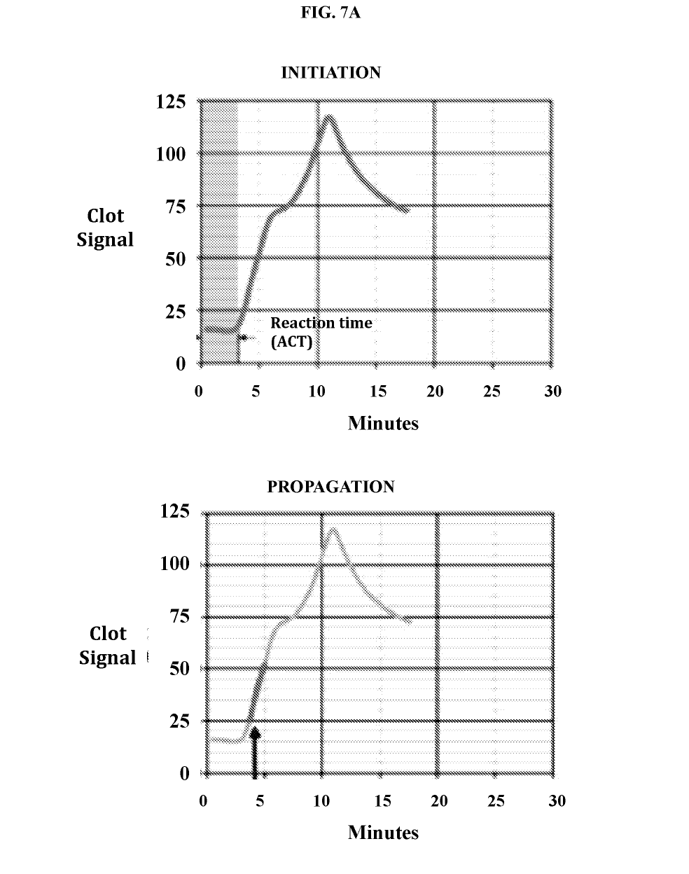

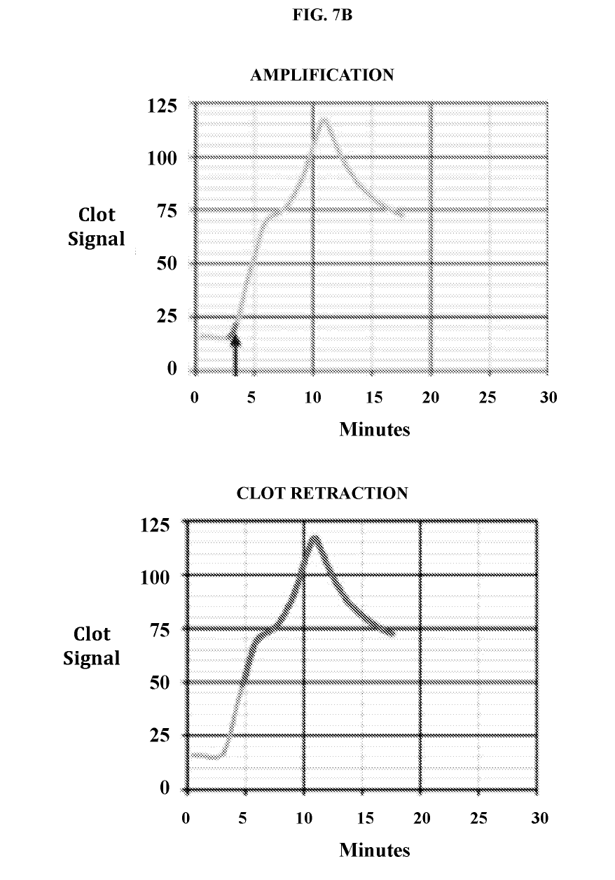

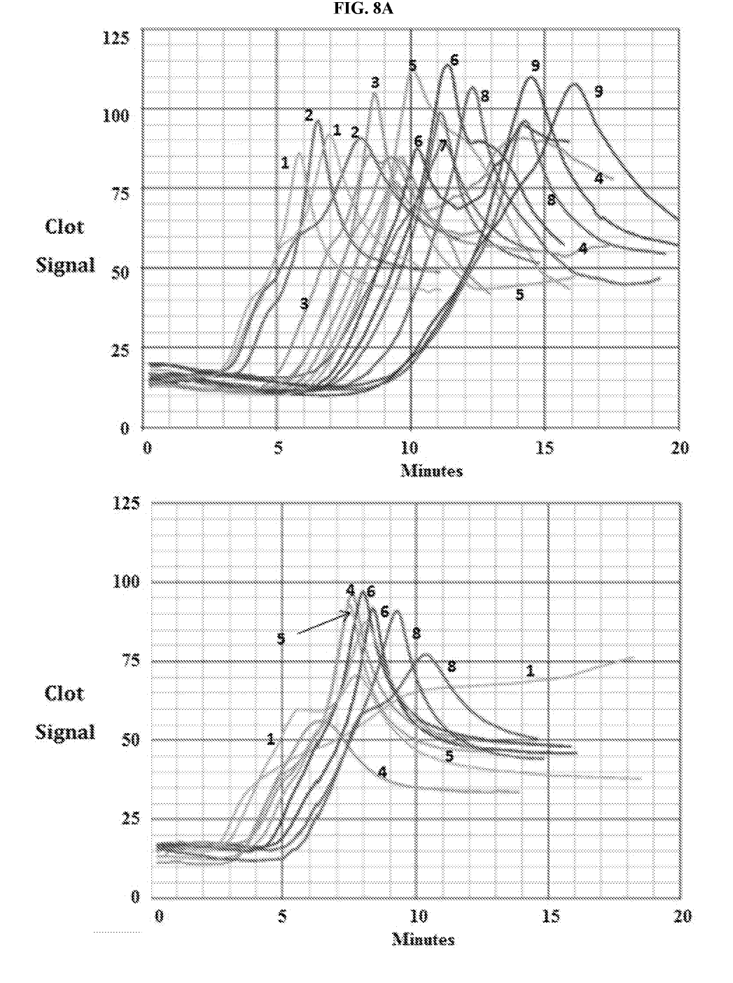

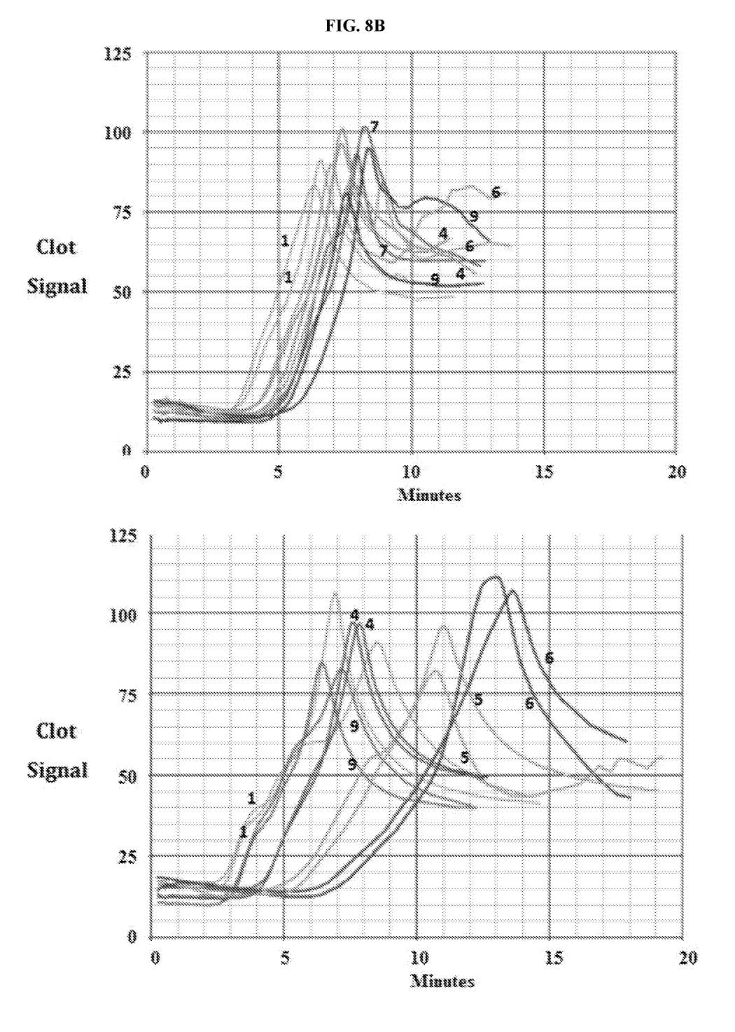

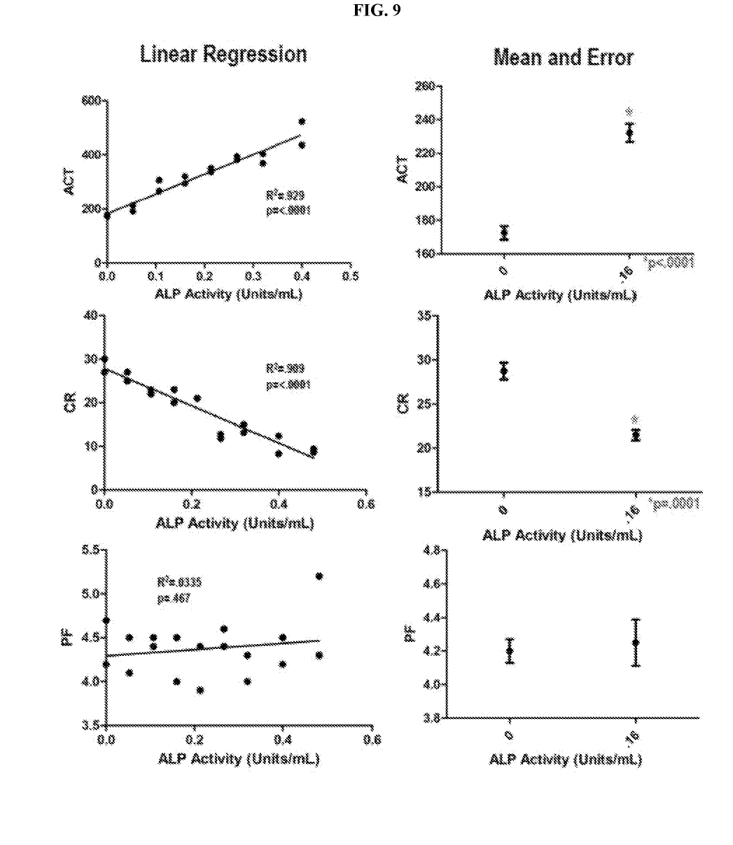

As demonstrated by FIGS. 6-7, 8A-8B, and 9, both types of AP resulted in an increase in ACT and a decrease in CR in a dose dependent response. The platelet function was not significantly affected. In regards to ACT, a significant positive correlation was noted between AP concentration and ACT (r²≥0.81 and p≤0.0003 for all samples). For CR, a significant negative correlation was noted between AP concentration and CR (r²≥0.64 and p≤0.0053 for all samples using the human liver AP). Trends in CR for human bone AP, although not as strong, were still significant (r²=0.41, p=0.0002). Individual donor responses varied to the same concentrations of AP, yet the overall trends were present in every donor.

The SCP2 revealed that increasing doses of AP significantly affects clot initiation, amplification, and propagation, increasing ACT and decreasing CR in healthy adults. Without wishing to be limited by any theory, these results can be due to the inhibition of the polyP molecules by AP, delaying clot formation. In certain non-limiting embodiments, AP's mechanism of protection involves prevention of a pro-inflammatory, pro-thrombotic state induced by polyP molecules during CV surgery and ultimately preventing organ injury and dysfunction.

### Example 4: CD73 after Infant Cardiopulmonary Bypass: Kinetics, Association with Clinical Outcomes, and Influence on Serum Adenosine Production Capacity

During cell death, ATP is released from cells, causing pathologic inflammation and ischemia reperfusion injury. Adenosine, a breakdown product of ATP works opposite ATP to inhibit inflammation. CD73 and alkaline phosphatase (AP) are the primary serum enzymes responsible for dephosphorylating extracellular ATP to adenosine. AP activity decreases following cardiopulmonary bypass (CPB), and soluble CD73 may increase after CPB. However, CD73 has never been directly assessed in pediatric subjects or following CPB.

In the present experiments, a sub-analysis of a large prospective cohort was performed, directed to serum samples of 85 infants <120 days old undergoing cardiothoracic surgery with CPB. CD73 levels were measured before CPB (after anesthesia induction and before first surgical incision) and during the rewarming phase of CPB. A subset of patients had samples available for analysis at 6 and 24 hours post-operatively. Conversion of 13C5-AMP to 13C5-adenosine was analyzed in a subset of serum samples using high performance liquid chromatography-mass spectrometry (HPLC-MS/MS). Multivariable analysis was used to model the relative contributions of CD73 and AP to AMP clearance capacity and short term clinical outcomes.

As demonstrated in Tables 12-17 and FIGS. 10-11, serum CD73 level persistently increases in infants following CPB and starts decreasing by 24 hrs. Subjects with higher pre-op CD73 levels are more sick as seen by their need for pre-op inotropic support and intubation. Subjects who were young, had single ventricle physiology, or required selective cerebral perfusion intra-operatively mounted lower post-op CD73 levels. Higher post-operative CD73 is independently associated with lower VIS at 24 hours and shorter ICU length of stay. CD73 and AP together predict serum adenosine production capacity. Nevertheless, AP acts as the dominant molecule. This study describes the kinetics of CD73 after infant cardiac surgery. Post-op, CD73 changes inversely to AP, which may serve to buffer the loss of serum adenosine that occurs with decreasing AP activity.

TABLE 12
Baseline Characteristics

| | N = 85 | |
|---|---|---|
| Age at surgery(days), median (range) | 13 | (2, 120) |
| Weight (kg), median (range) | 3.5 | (2.1, 7.9) |
| Aristotle Score - Comprehensive; median (range) | 9.5 | (3, 16) |
| Single ventricle physiology, n (%) | 27 | (32.1%) |
| Pre-operative intubation, n(%) | 30 | (35%) |
| Pre-operative inotropic support, n(%) | 12 | (14.1%) |
| Pre-operative steroid, n(%) | 36 | (43%) |
| Cardiopulmonary bypass time (minutes), median | 144 | (54, 399) |
| (range) | | |
| Cross-clamp time (minutes), median (range) | 76 | (0, 241) |
| Deep hypothermic circulatory arrest (minutes), | 0 | (0, 154) |
| median (range) | | |
| Selective cerebral perfusion (minutes), median | 0 | (0, 82) |
| (range) | | |

TABLE 13
Baseline characteristics and pre-operative CD73 levels

| | Pre-Operative CD73 level | | | | |
|---|---|---|---|---|---|
| | <50% | | >=50% | | P |
| Baseline Characteristics | n = 43 | | n = 42 | | Value |
| Age (days), median (range) | 8 | (2, 120) | 14 | (2, 109) | NS |
| Weight (kg), median (range) | 3.8 | (2.6, 7.4) | 3.3 | (2.1, 7.9) | NS |
| Aristotle Score - | 9 | (3.0, 14.5) | 10 | (3.0, 16.0) | NS |
| Comprehensive, median | | | | | |
| (range) | | | | | |
| Single ventricle physiology, | 15 | (34.9%) | 12 | (29.3%) | NS |
| n (%) | | | | | |
| Pre-operative intubation, n(%) | 7 | (16.3%) | 23 | (54.8%) | <0.001 |
| Pre-operative inotropic | 2 | (4.7%) | 10 | (23.8%) | <0.05 |
| support, n(%) | | | | | |
| Pre-operative steroid, n(%) | 22 | (52.4%) | 26 | (61.9%) | NS |

TABLE 14
Subject characteristics and post-operative CD73 levels

| | Post-Operative CD73 level | | | | |
|---|---|---|---|---|---|
| | <50% | | >=50% | | P |
| Subject Characteristics | n = 43 | | n = 43 | | Value |
| Age (days), median (range) | 6 | (2, 120) | 32 | (4, 118) | <0.01 |
| Weight (kg), median (range) | 3.3 | (2.1, 7.4) | 3.6 | (2.1, 7.9) | NS |
| Aristotle Score - Comprehensive, median (IQR) | 10 | (8.0, 14.5) | 9 | (6.3, 11.0) | NS |
| Single ventricle physiology, n (%) | 19 | (45.2%) | 8 | (18.6%) | <0.01 |
| Pre-operative intubation, n(%) | 13 | (30.2%) | 17 | (39.5%) | NS |
| Pre-operative inotropic support, n(%) | 5 | (11.6%) | 7 | (16.3%) | NS |
| Pre-operative steroid, n(%) | 28 | (65.1%) | 21 | (50%) | NS |
| Cardiopulmonary bypass time (minutes), median (IQR) | 154 | (116, 201) | 122 | (88, 204) | NS |
| Cross-clamp time (minutes), median (range) | 79 | (68, 96) | 74 | (49, 115) | NS |
| Deep hypothermic circulatory arrest (minutes), | 2 | (0, 10) | 0 | (0, 6) | NS |
| median (range) | | | | | |
| Selective cerebralperfusion (minutes), median (range) | 0 | (0, 46) | 0 | (0, 0) | <0.01 |

TABLE 15
Post-operative CD73 and clinical outcomes

| | Post-Operative CD73 level | | | | |
|---|---|---|---|---|---|
| | <50% | | >=50% | | P |
| Subject Characteristics | n = 43 | | n = 43 | | Value |
| VIS 6 hour post- | 10.6 | (4.4) | 9.5 | (4.0) | NS |
| operative, mean (std) | | | | | |
| VIS 24 hour post- | 10.3 | (5.6) | 7.3 | (4.5) | <0.01 |
| operative, mean (std) | | | | | |
| Intubation, median | 50.9 | (24.7, 93.4) | 35.8 | (20.1, 67.8) | 0.06 |
| (IQR) | | | | | |
| Days of ICU stay | 7.0 | (4.5, 17.0) | 4.0 | (3.0, 7.0) | <0.05 |

TABLE 16
Multivariable Modeling of CD73 and AP with clinical outcomes

| | | | ICU length of stay | | |
|---|---|---|---|---|---|
| VIS at 24 hours | | | | Change in ICU | |
| | Change in 24 hr VIS per | | | length of stay per | |
| Independent | 10 unit increase in | | Independent | 10 unit increase in | |
| variable | independent variable | p-value | variable | independent variable | p-value |
| Pre-op CD73 | 2% | <0.05 | Pre-op CD73 | 5% | <0.01 |
| Post-op CD73 | −2% | <0.005 | Post-op CD73 | −6% | <0.005 |
| Post-op AP | −3% | <0.005 | Post-op AP | −10% | <0.005 |

TABLE 17
Multivariable analysis of ex-vivo adenosine production

| Independent | Change in adenosine per 10 unit increase in | |
|---|---|---|
| variable | independent variable | p-value |
| Post-op CD73 | 0.2 | 0.15 |
| Post-op AP | 0.7 | <0.0001 |

### Example 5: Alkaline Phosphatase and Endotoxin Activity after Infant Cardiopulmonary Bypass

AP detoxifies endotoxin in vitro (dephosphorylation of phosphate groups on the highly conserved lipid A moiety), but this action of AP has not been studied in human disease. Endotoxemia occurs frequently after infant cardiopulmonary bypass (CPB) and may adversely affect post-operative care. Loss of AP activity after infant cardiac surgery is associated with increased post-operative support requirements.

In certain embodiments, AP clears endotoxin after infant cardiac surgery. In order to study this process, the present studies assessed association between low serum AP activity and endotoxemia, as well as determined effect of ex vivo administration of human liver AP on reduction of serum Endotoxin Activity Assay (EAA).

For this study, the prospective cohort studied comprised 62 infants <120 days of age undergoing cardiothoracic surgery with CPB. AP activity and EAA were measured pre-operatively, during rewarming, and 24 hours after arrival in the cardiac intensive care unit. Low pre-op AP activity was prospectively defined as ≤150 U/L and low rewarming AP activity defined as ≤80 U/L based on pilot data. EAA was measured via a commercially available assay (Spectral Medical Inc, Toronto, Calif.), using the following classification: >0.6=high endotoxin burden; 0.4-0.59=moderate endotoxin burden; and <0.39=low endotoxin burden. In a subset of 22 subjects, EAA was measured with and without addition of 1,500 U/L of AP to pre-operative and rewarming serum samples.

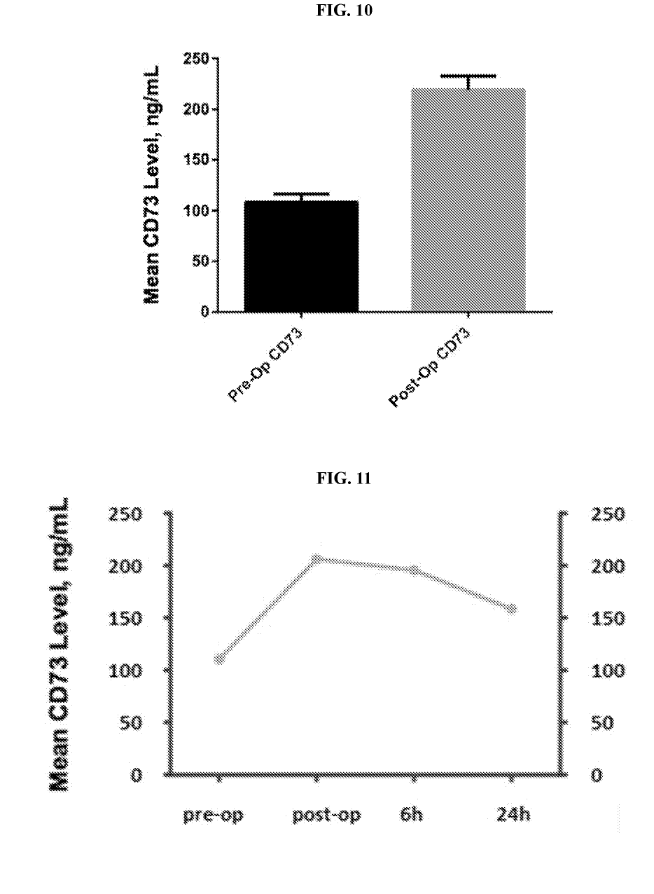

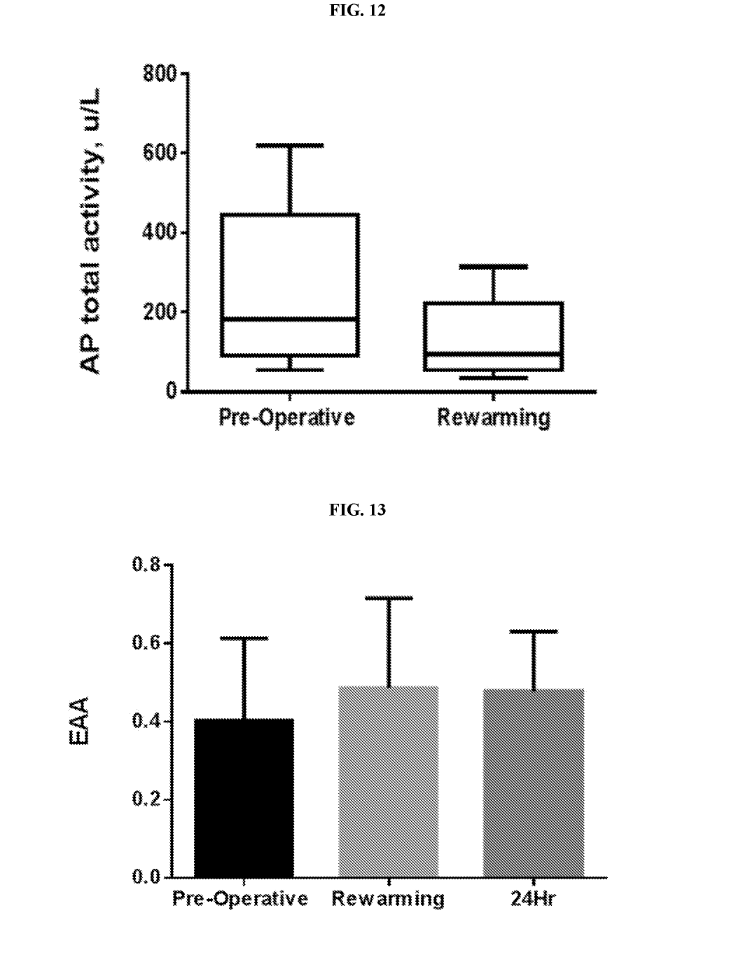

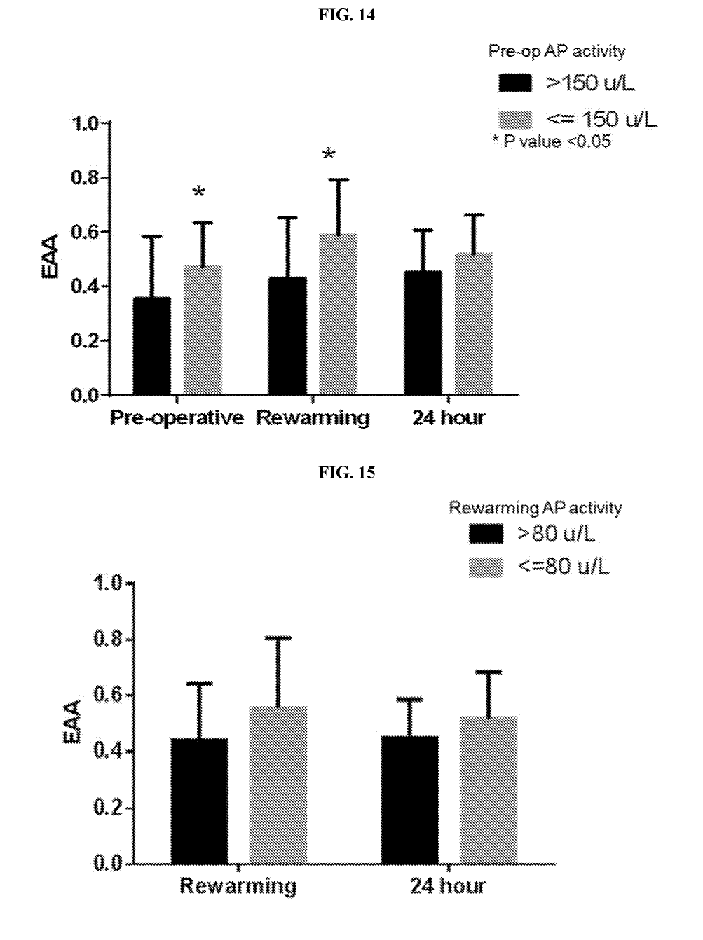

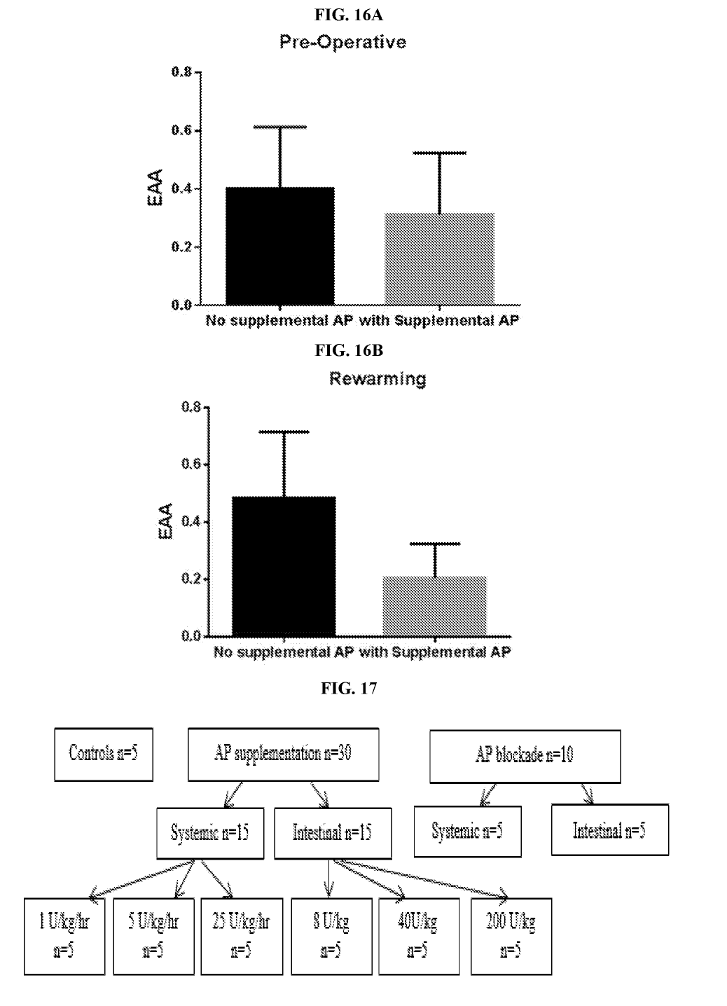

As demonstrated by Tables 18-19 and FIGS. 12-15 and 16A-16B, AP activity decreases, while EAA increases following infant cardiothoracic surgery with cardiopulmonary bypass. Low pre-operative AP is associated with higher EAA. Low post-operative AP shows a similar trend but does not reach statistical significance. In one aspect, ex vivo addition of liver AP markedly reduces serum EAA.

TABLE 18
Demographics and baseline characteristics

| | | |
|---|---|---|
| Male sex (%) | 33 | (53.2%) |
| Age at surgery, days; median[range] | 10 | (2, 118) |
| Preterm (%) | 8 | (13.1%) |
| Weight; median[range] | 3.7 | (2.1, 7.9) |
| Pre-operative mechanical ventilation (%) | 19 | (32.8%) |
| Pre-operative inotropic support (%) | 3 | (4.8%) |
| Aristotle Score-Comprehensive; median[range] | 9 | (3, 16) |
| Aristotle Score-Basic; Median[range] | 9 | (3, 14.5) |
| Cardiopulmonary bypass time, minutes; median[range] | 157 | (0, 399) |
| Cross-clamp time, minutes; median[range] | 77 | (0, 205) |
| Deep hypothermic circulatory arrest, minutes; | 0 | (0, 154) |
| median[range] | | |
| Selective cerebral perfusion, minutes; media[range] | 0 | (0, 82) |
| Single ventricle physiology (%) | 19 | (31.2%) |
| Perioperative steroid administration (%) | 32 | (55.2%) |

TABLE 19
Effect of in vivo liver AP supplementation
(average change in EAA per subject)

| | Decrease in | % Decrease in | |
|---|---|---|---|
| | EAA: mean | EAA: mean | |
| | [SD] | [SD] | p value |
| Pre-operative | 0.12 [0.14] | 29% [35] | <0.001 |
| samples | | | |
| Post-operative | 0.26 [0.19] | 51% [18] | <0.0001 |
| samples | | | |

### Example 6: Therapeutic Intervention for Deep Hypothermic Circulatory Arrest (DHCA) in a Pig

Table 11 illustrates certain markers that are associated with organ dysfunction and/or injury in a subject subjected to CPB. Such markers include creatinine (which reports on kidney dysfunction and/or injury), iFABP (intestinal fatty acid binding protein, which reports on intestinal dysfunction and/or injury), lactate (which reports on general anaerobic metabolism), and NT-proBNP (N-terminal pro b-type natriuretic peptide, which reports on cardiac function).

A model interventional study was performed in infant piglets (5-10 kg) to assess the effect of administering biAP to subjects that had been subjected to cardiopulmonary bypass (CPB) with DHCA. The model set is illustrated in FIG. 17. Briefly, the subjects were initially subjected to neck cannulation (internal jugular and common carotid), then cooled to 22° C. and kept at that temperature for 75 minutes under complete circulatory arrest. The subjects were then rewarmed to standard body temperature on CPB and kept intubated for 4 hours. Sham-only subject were subjected only to the neck cannulation (not the CPB or drug administration). DHCA control subjects were subjected to CPB and DHCA without receiving BiAP. High dose BiAP subjects were exposed to CPB with DHCA and high dose biAP (75 U/kg iv bolus followed by continuous infusion of 25 U/kg/hr).

Table 20 summarizes AP activity in the study. DHCA control subjects showed decreasing AP activity after the surgical procedure, while animals treated with BiAP showed much increased AP activity as expected.

TABLE 20
AP Activity
Alkaline Phosphatase Activity (U/L)

| | Study ID | | CPB | | |
|---|---|---|---|---|---|
| | Number | Induction | Initiation | Rewarming | Euthanasia |
| Sham-only | | 129 | 115* | 102* | 102 |
| DHCA | 8 | 126 | 108 | 96 | 113 |
| (90 min) | | | | | |
| DHCA | 9 | 139 | 93 | 80 | 112 |
| (75 min) | | | | | |
| DHCA | 12 | 76 | 55 | 54 | 81 |
| (75 min) | | | | | |
| High Dose | 10 | 97 | 517 | 935 | 445 |
| BiAP | | | | | |
| High Dose | 13 | 184 | 317 | 447 | 437 |
| BiAP | | | | | |

Table 21 illustrates amount of cardiovascular support required (summarized by the vasoactive inotropic score) for tested subjects from the time of rewarming after DHCA to euthanasia four hours after rewarming and separation from CPB.

TABLE 21
Cardiovascular Physiology
Vasoactive Inotropic Score

| | Study ID | | | | | |
|---|---|---|---|---|---|---|
| | Number | Rewarming | 1 hr | 2 hr | 3 hr | Euthanasia |
| Sham-only | | 0 | | 0 | 0 | 0 |
| DHCA | 8 | 12 | 50 | 38.5 | 50 | 45 |
| (90 min) | | | | | | |
| DHCA | 9 | 12 | 20 | 23 | 23 | 23 |
| (75 min) | | | | | | |
| DHCA | 12 | 19 | 19 | 19 | 19 | 19 |
| (75 min) | | | | | | |
| High Dose | 10 | 12 | 15 | 15 | 15 | 15 |
| BiAP | | | | | | |
| High Dose | 13 | 13.5 | 8.5 | 8.5 | 8.5 | 8.5 |
| BiAP | | | | | | |

Table 22 illustrates the measured PaO2/FiO2 ratios for the tested subjects. PaO2 is the partial pressure of oxygen dissolved in the subject's blood, expressed in mmHg. FiO2 (fraction of inspired oxygen) is the fraction or percentage of oxygen gas in the gas being inspired by the subject. PaO2/FiO2 is the ratio of partial pressure arterial oxygen and fraction of inspired oxygen (sometimes called the Carrico index), and is a comparison between the oxygen level in the blood and the oxygen concentration that is breathed. The PaO2/FiO2 ratio is an indication of any problems with how the lungs transfer oxygen to the blood. A PaO2/FiO2 ratio ≤200 allows for diagnosis of acute respiratory distress syndrome, while a ratio of 200-300 allows for diagnosis of acute lung distress. A PaO2/FiO2 ratio ≥300 indicates normal lung function.

As shown in Table 22, the DHCA control subjects showed lung injury, with PaO2/FiO2 ratios dipping into the 200s and even 100s. On the other hand, subjects treated with high dose BiAP showed throughout the study consistently higher PaO2/FiO2 ratios than the DHCA control subjects.

TABLE 22
PaO2/FiO2

| | Study ID | | | | | |
|---|---|---|---|---|---|---|
| | Number | Induction | 1 hr | 2 hr | 3 hr | Euthanasia |
| Sham-only | | 367 | 325 | 386 | 382 | 364 |
| DHCA | 8 | 358 | 211 | 196 | 152 | 140 |
| (90 min) | | | | | | |
| DHCA | 9 | 337 | 287 | 241 | 226 | 225 |
| (75 min) | | | | | | |
| DHCA | 12 | 249 | 215 | 219 | 223 | 187 |
| (75 min) | | | | | | |
| High Dose | 10 | 382 | 215 | 248 | 312 | 269 |
| BiAP | | | | | | |
| High Dose | 13 | 329 | 284 | 259 | 234 | 308 |
| BiAP | | | | | | |

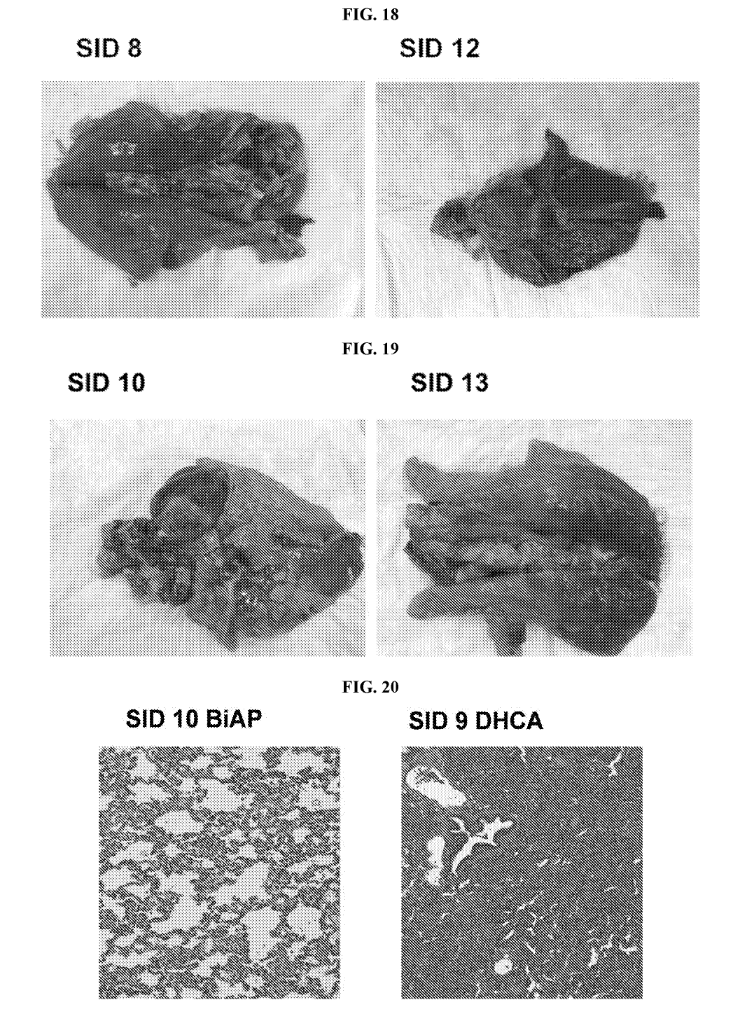

FIG. 18 illustrate non-limiting gross pathology lung samples obtained according to Example 6. The samples are derived from subjects subjected to DHCA without therapeutic intervention.

FIG. 19 illustrate non-limiting gross pathology lung samples obtained according to Example 6. The samples are derived from subjects subjected to DHCA and treated with BiAP (25 U/kg/hr).

FIG. 20 illustrate non-limiting gross pathology lung samples obtained according to Example 6. Sample SID 10 is from a subject subjected to DHCA and treated with BiAP (25 U/kg/hr). Sample SID 9 is from a subject subjected to DHCA without therapeutic intervention (control sample). Sample SID 10 displayed much higher lung tissue air expansion, reduced inflammatory cell infiltrate, and reduced interstitial thickening than sample SID 9.

Table 23 illustrates urine analysis for selected subjects within the study. The DHCA control subjects showed signs of significant kidney damage, as evidenced by the presence of high amounts of protein and blood in the urine. On the other hand, subjects treated with high dose of BiAP showed only trace amounts of protein and low amounts of blood in the urine, comparable to the sham-only subjects.

TABLE 23
Urinalysis

| | Specific | | | |
|---|---|---|---|---|
| | Gravity | pH | Protein | Blood |
| Sham | 1.025 | 5 | trace | none |
| DHCA (90 min) | 1.025 | 5 | 100 (++) | 250 |
| DHCA (75 min) | 1.020 | 5 | 100 (++) | 250 |
| High Dose BiAP | 1.015 | 7 | trace | 50 |

The results presented herein show that administration of AP to subjects that had been subjected to CPB help treat or preventing inflammation or organ dysfunction in the subjects, as compared to control subjects that undergo no therapeutic intervention.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inhibiting or reducing blood coagulation in a human subject, the method comprising administering to the subject a therapeutically effective amount of isolated human alkaline phosphatase (AP), or a biologically effective fragment, derivative, conjugate, or recombinant form thereof.

2. A method of treating or preventing inflammation or organ dysfunction in a human subject, the method comprising administering to the subject a therapeutically effective amount of isolated human AP, or a biologically effective fragment, derivative, conjugate, or recombinant form thereof.

3. A method of reducing levels of extracellular adenine nucleotide in a human subject, the method comprising administering to the subject a therapeutically effective amount of isolated human AP, or a biologically effective fragment, derivative, conjugate, or recombinant form thereof.

4. A method of reducing levels of endotoxins in the blood of a human subject, the method comprising administering to the subject a therapeutically effective amount of human AP, or a biologically effective fragment, derivative, conjugate, recombinant form thereof.

5. A method of treating or preventing organ dysfunction in a human subject, the method comprising administering to the subject a therapeutically effective amount of isolated bovine intestinal AP, or a biologically effective fragment, derivative, conjugate, or recombinant form thereof.

6. The method of claim 1, wherein activated clotting time (ACT) in the subject is increased.

7. The method of claim 1, wherein clot rate (CT) in the subject is decreased.

8. The method of claim 2, wherein the human AP is at least one selected from the group consisting of intestinal, placental, placental-like, and tissue non-specific.

9. The method of claim 2, wherein the human AP is at least one selected from the group consisting of intestinal, placental, and tissue non-specific.

10. The method of claim 2, wherein the human AP is tissue non-specific.

11. The method of claim 8, wherein the human tissue non-specific AP is at least one selected from the group consisting of liver, bone, and kidney.

12. The method of claim 8, wherein the human tissue non-specific AP is human liver or bone AP.

13. The method of claim 8, wherein the human tissue non-specific AP is human liver AP.

14. The method of claim 1, wherein the subject has undergone or is undergoing cardiovascular surgery.

15. The method of claim 14, wherein the subject has undergone or is undergoing cardiopulmonary bypass (CPB).

16. The method of claim 14, wherein the subject has undergone or is undergoing extracorporeal membrane oxygenation (ECMO).

17. The method of claim 1, wherein the subject is an infant of about 120 days or less of age.

18. The method of claim 1, wherein the AP is administered in a dose varying from about 4 U/kg to about 150 U/kg.

19. The method of claim 1, wherein the administration allows the subject to maintain a blood AP activity of at least about 80 U/L.

20. The method of claim 1, wherein the administration allows the subject to maintain a blood AP activity of at least about 100-1,000 U/L.
